(19) United States
(12) Patent Application Publication
(10) Pub. No.: US 2011/0190513 A1
(43) Pub. Date: Aug. 4, 2011

(54) METHODS, COMPOSITIONS AND SYSTEMS FOR BIOSYNTHETIC BIO-PRODUCTION OF 1,4-BUTANEDIOL

(76) Inventor: Michael D. Lynch, Boulder, CO (US)

(21) Appl. No.: 13/002,941

(22) PCT Filed: Jul. 8, 2009
(86) PCT No.: PCT/US09/49973

§ 371 Date: Apr. 7, 2011

Related U.S. Application Data
(60) Provisional application No. 61/134,214, filed on Jul. 8, 2008.

(51) Int. Cl. C07D 307/33; C12N 1/00; C12P 7/18; C12M 1/00; C07D 307/08; C07C 269/00; C07C 67/08

(52) U.S. Cl. 549/295; 435/243; 435/158; 435/289.1; 549/509; 560/1

(57) ABSTRACT

Three biosynthetic pathways are disclosed for microorganism bio-production of 1,4-Butanediol from various carbon sources. Exemplary methods are provided. The recombinant microorganisms comprising any of these 1,4-Butanediol biosynthesis pathways may also comprise genetic modifications directed to improved tolerance for 1,4-Butanediol.

### CROSS-REFERENCE

This application claims the benefit of U.S. Provisional Application No. 61/134,214, filed Jul. 8, 2008, which application is incorporated herein by reference in their entirety.

### REFERENCE TO A SEQUENCE LISTING

This patent application provides a paper copy of sequence listings that are to be provided on compact disk in appropriate format in a later filing.

### FIELD OF THE INVENTION

The present invention relates to methods, systems and compositions, including genetically modified microorganisms, adapted to produce 1,4-butanediol (“1,4-BDO”). In various embodiments these organisms are genetically modified so that an elevated titer of 1,4-BDO is achieved, such as in industrial bio-production systems based on microbial biosynthetic activity. In other embodiments these organisms are genetically modified so that an elevated production rate of 1,4-BDO is achieved, such as in industrial bio-production systems based on microbial biosynthetic activity.

### BACKGROUND OF THE INVENTION

1,4-butanediol (“1,4-BDO”) is a chemical of value to manufacturing industries worldwide. Its conversions and uses are well known the chemical engineers, polymer scientists and technicians, and the like. Generally 1,4-BDO is used as an industrial solvent and also in the manufacture of some types of plastics and fibers. It has similar industrial applications as 1,3-propanediol and is a precursor for butyrolactone and tetrahydrofuran.

Among its many uses is its use in polybutylene terephthalate, an industrial polymer that comprises a terephthalic acid component and a 1,4-BDO component. Polybutylene terephthalate is widely used in injection molded articles such as automotive parts, electric or electronic parts, and precision machine parts as one of engineering plastics having mechanical properties and heat resistance, which can be a substitute for metallic materials. In recent years, has also been widely used in fields including films, sheets, monofilaments, and fibers because of its excellent properties.

Given 1,4-BDO's many valued uses as a chemical commodity in various industrial chemical reactions and ultimately for various products, there is concern about its cost and ultimate supply prospects in view of generally downwardly shifting supplies of petroleum hydrocarbons. This is because petroleum hydrocarbons currently are the primary source for chemical 1,4-BDO production.

Thus, there is recent interest in biosynthetic alternatives for production of 1,4-BDO. Notwithstanding developments in this arena, there remains a need in the art for cost-effective and reliable biosynthetic approaches to the industrial-scale production of 1,4-BDO.

### BRIEF DESCRIPTION OF THE DRAWINGS

FIG. 1 provides a summary of metabolic pathways for production of 1,4-BDO from sugars. FIG. 1 is provided on two sheets each providing a partial view of these pathways, and are meant to be combinable to provide a single view of these pathways.

FIG. 2 provides a calibration curve for 1,4-BDO.

### DETAILED DESCRIPTION OF EMBODIMENTS OF THE INVENTION

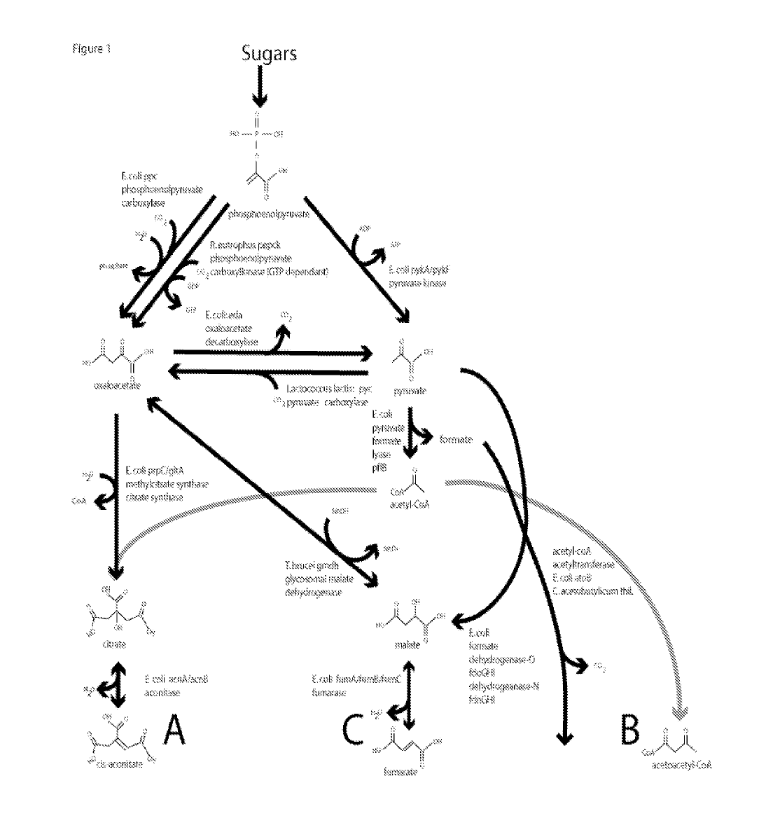

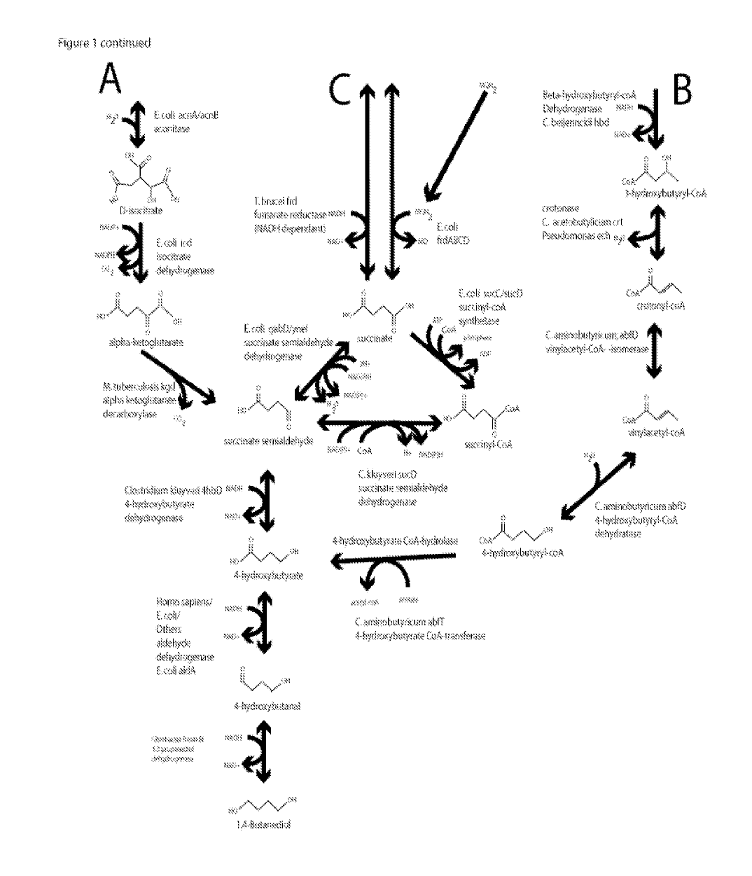

One general aspect of the present invention pertains to microbial biosynthetic pathways for the production of 1,4-BDO from common carbon sources other than petroleum hydrocarbons. A number of alternative microbial biosynthetic pathways for production of 1,4-BDO are shown in FIG. 1. FIG. 1 also describes each enzyme choice for each step, providing alternative choices for some steps, the respective choice including an indication of the organism source for a respective enzyme. These descriptions are part of the present disclosure and may be incorporated into the detailed description and/or claims of a later filing of a patent application claiming priority hereto.

The enzyme functions to complete a functional microbial biosynthetic pathway for 1,4-BDO production may be provided in a microorganism of interest by use of a plasmid, or other vector capable of and adapted to introduce into that microorganism a gene encoding for a respective enzyme having a desired respective function. Other techniques standard in the art allow for the integration of DNA allowing for expression of these enzymatic functions into the genome of numerous microorganisms. These techniques are widely known and used in the art, and generally may follow methods provided in Sambrook and Russell, Molecular Cloning: A Laboratory Manual, Third Edition 2001 (volumes 1-3), Cold Spring Harbor Laboratory Press, Cold Spring Harbor, N.Y. (“Sambrook and Russell”).

In cases where introduction of more than one gene is required for a particular microorganism, a single vector may be engineered to provide more than one such gene. The two or more genes may be designed to be under the control of a single promoter (i.e., a polycitronic arrangement), or may be under the control of separate promoters and other control regions.

Accordingly, based on the high level of skill in the art and the many molecular biology and related recombinant genetic technologies known to and used by those of skill in the art, there are many approaches to obtaining a recombinant microorganism comprising a 1,4-BDO biosynthetic pathway capable of producing 1,4-BDO from a desired carbon source. The examples provided below are not meant to be limiting of the wide scope of possible approaches to make biological compositions comporting with the present invention, wherein any of those approaches may, without undue experimentation, result in composition(s) that may be used to achieve substantially the same solution as disclosed herein to obtain a desired biosynthetic industrial production of 1,4-BDO.

Referring to FIG. 1, three basic 1,4-BDO biosynthetic pathways are depicted. These may be interrelated with alternatives and variations as indicated in the figure and/or as described and/or referred to herein. In various embodiments, any of a wide range of sugars, such as sucrose, glucose, xylose, cellulose or hemixellulose (this list not meant to be limiting), are provided to a microorganism, such as in an industrial system comprising a reactor vessel in which a defined media (such as a minimal salts media including M9 minimal media, Potassium Sulfate minimal media, yeast synthetic minimal media and many others or variations of these), an inoculum of the microorganism comprising one of the 1,4-BDO biosynthetic pathways, and the sugar as a carbon source may be combined. The sugar enters the cell and is catabolically metabolized by well-known and common metabolic pathways to yield common metabolic intermediates, including phosphoenolpyruvate (PEP). (See Molecular Biology of the Cell, 3^{rd} Ed., B. Alberts et al. Garland Publishing, New York, 1994, pp. 42-45, 66-74, incorporated by reference for the teachings of basic metabolic catabolic pathways for sugars; Principles of Biochemistry, 3^{rd} Ed., D. L. Nelson & M. M. Cox, Worth Publishers, New York, 2000, pp 527-658, incorporated by reference for the teachings of major metabolic pathways; and Biochemistry, 4^{th} Ed., L. Stryer, W. H. Freeman and Co., New York, 1995, pp. 463-650, also incorporated by reference for the teachings of major metabolic pathways.).

A first 1,4-BDO biosynthetic pathway, labeled “A,” metabolizes PEP to oxaloacetate using, for example (not to be limiting) a phosphoenolpyruvate carboxylase such as of E. coli (ppc) or GTP-dependent phosphoenolpyruvate carboxylase such as of R. eutrophus (pepck). This step consumes a carbon dioxide molecule, adding it to PEP to yield oxaloacetate, and also yields, for the stated enzymes, phosphate or GTP respectively (see FIG. 1 for other details). Oxaloacetate can also be obtained from the metabolite pyruvate such as by the enzyme pyruvate carboxylase such as from L. lactis (pepck). In the next step of biosynthetic pathway A oxaloacetate combines with acetyl-CoA to form citrate. Either of the enzymes methylcitrate synthase or citrate synthase, such as from E. coli (prpC, gltA) may be used to achieve this step. As shown in FIG. 1, for each oxaloacetate a water molecule is consumed and one CoA molecule is released. The acetyl CoA may be provided in the cell by any of the pathways indicated in FIG. 1 that result in its production, and also via metabolic pathways described in the published resources incorporated by reference in the previous paragraph.

Citrate then is converted to cis-aconitrate such as by using aconitrase from E. coli (acnA or acnB). Aconitase, such as from E. coli, also converts cis-aconitrate to D-isocitrate, which is converted to alpha-ketoglutarate such as by isocitrate dehydrogenase from E. coli (icd).

Then alpha-ketoglutarate decarboxylase from M. tuberculosis (kgd) converts alpha-ketoglutarate to succinate semialdehyde. The preparation of a vector comprising the gene for this enzyme is described below. It is noted that other analogous genes may be used, and this example, particularly as to the source or specific methods, is not meant to be limiting.

Succinate semialdehyde is converted to 4-hydroxybutyrate, such as by a 4-hydroxybutyrate dehydrogenase from Clostridium kluyveri (4hbD). 4-hydroxybutyrate is converted to 4-hydroxybutanal by an aldehyde dehydrogenase, which may be selected from a number of available such enzymes from E. coli, H. sapiens, or other species. Finally, 4-hydroxybutyrate is converted to 1,4-BDO by a 1,3-propanediol dehydrogenase such as from Citrobacter freundii (dhaT). The particular enzymes recited are not to be limiting.

It is noted that PCT/US2001/022834, having an International filing date of Jul. 20, 2001 and a priority date of Jul. 20, 2000, which is directed to microbial production of polyhydroxyalkanoates, discloses that a diol oxidoreductase converts 1,4-BDO to 4-hydroxybutyraldehyde. This is then converted to 4-hydroxybutyrate by an aldehyde dehydrogenase. Although demonstrating conversion in a direction opposite to the above approach, this patent publication supports the feasibility of use of an aldehyde dehydrogenase for the purpose intended herein.

A second 1,4-BDO biosynthetic pathway, labeled “B,” may be considered to begin with the enzymatic condensation of two acetyl-CoA molecules to acetoacetyl-CoA. This reaction may be catalyzed by an acetyl-CoA acetyltransferase, such as from E. coli (atoB) or from C. acetobutylicum (thiL). As shown in FIG. 1, and further as known to those skilled in the art and referred to above, acetyl CoA may be supplied by one or more of a number of metabolic conversions derived from a number of major (and minor) pathways.

Acetoacetyl-CoA is converted to 3-hydroxybutyryl-CoA such as by a reaction catalyzed by a β-hydroxybutyryl-CoA dehyrogenase from C. beijerinckii (hbd). 3-hydroxybutyryl-CoA is converted to crotonyl-CoA such as by a crotonase, such as from C. acetobutylicum (crt) or from Pseudomonas spp. (ech). Crotonyl-CoA is converted to vinylacetyl-CoA, such as by vinylacetyl-CoA-A-isomerase, for example from C. acetobutylicum (abfD). The same enzyme, demonstrating 4-hydroxybutyryl-CoA dehydratase activity, also has enzymatic activity to convert vinylacetyl-CoA to 4-hyroxybutyryl-CoA. 4-hyroxybutyryl-CoA is converted to 4-hyroxybutyrate, such as by a 4-hydroxybutyrate-CoA-transferase also from C. acetobutylicum (abfT).

Thereafter biosynthetic pathway B comprises the following two steps as described above for biosynthetic pathway A. 4-hydroxybutyrate is converted to 4-hydroxybutanal by an aldehyde dehydrogenase, which may be selected from a number of available such enzymes (e.g., adh) from E. coli, H. sapiens, or other species. Finally, 4-hydroxybutyrate is converted to 1,4-BDO by 1,3-propanediol dehydrogenase such as from Citrobacter freundii (dhaT).

A third 1,4-BDO biosynthetic pathway, labeled “C,” may begin similarly to pathway A above, by metabolizing PEP to oxaloacetate using, for example (not to be limiting) phosphoenolpyruvate carboxylase (ppc) of E. coli or GTP-dependent phosphoenolpyruvate carboxylase (pepck) of R. eutrophus (see above and FIG. 1 for other details). Oxaloacetate can also be obtained from the metabolite pyruvate such as by the enzyme pyruvate carboxylase from L. lactis (pyc). In the next step of these initial conversions for biosynthetic pathway C oxaloacetate is converted to malate, such as by a glycosomal malate dehydrogenase from T. brucei (gmdh), as shown in FIG. 1. The NADH may be replenished by normal cellular metabolism or by engineering NADH producing pathways into the host, in particular NADH producing pathways.

Malate is converted to fumarate, such as by any one or more of E. coli's known fumarase isozymes, fumA, fumB, and fumC, releasing a water molecule. Then a fumarate reductase enzyme, such as the NADH-dependent fumarate reductase of T. brucei (frd), or the E. coli fumarate reductase encoded by the frdABCD operon converts fumarate to succinate. The latter may receive its reducing equivalents as described below.

Thereafter succinate is enzymatically converted to succinate semialdehyde, such as by the succinate semialdehyde dehydrogenase from E. coli (encoded by either gabD or yneI genes). Thereafter biosynthetic pathway C comprises the following three steps as described above for biosynthetic pathway A.

Succinate semialdehyde is converted to 4-hydroxybutyrate, such as by 4-hydroxybutyrate dehydrogenase from Clostridium kluyveri (4hbD). 4-hydroxybutyrate is converted to 4-hydroxybutanal by an aldehyde dehydrogenase, which may be selected from a number of available such enzymes encoded by nucleic acid sequences (e.g., adh) from E. coli, H. sapiens, or other species. Finally, 4-hydroxybutyrate is converted to 1,4-BDO by a 1,3-propanediol dehydrogenase such as from Citrobacter freundii (dhaT).

Further as to the third pathway, C, there are a number of other initial conversions variations leading to malate, some of them shown in FIG. 1. That is, malate may be derived from PEP via pyruvate, the latter reaction catalyzed such as by a pyruvate kinase from E. coli (pykA or pykF isozymes), and then from pyruvate to malate such as by malic enzymes encoded by genes including maeA from E. coli (Alternatively PEP can be enzymatically converted to oxaloacetate, such as as described above, and oxaloacetate may then be converted into pyruvate (such as by an oxaloacetate decarboxylase from E. coli (eda). The pyruvate would then convert to malate such as described immediately above. These comprise alternative initial conversions to the above-described initial conversion comprising oxaloacetate to malate (such as by a glycosomal malate dehydrogenase, such as from T. brucei (gmdh)).

Further, as illustrated in FIG. 1, a further downstream alternative of biosynthetic pathway C is that succinate may be converted to succinyl-CoA, and then at least a portion of the succinyl-CoA is converted to succinate semialdehyde. The respective enzymes are succinyl-CoA synthetase, such as from E. coli (sucC and sucD, encoding, respectively, β- and α-subunits) and succinate semialdehyde dehydrogenase, such as from C. kluyveri (sucD). Without being bound to a particular theory, there may be a thermodynamic advantage to this approach, which links progression through the metabolic pathway to hydrolysis of ATP.

Based on the initial conversions variations first noted above, for biosynthetic pathway C, and from FIG. 1, it is apparent that at least on upstream variation also exists for biosynthetic pathway A. That is, oxaloacetate may be obtained less directly than described above, from PEP, such as from PEP to pyruvate, and then to oxaloacetate, the latter by an enzyme such as pyruvate carboxylase, for example from Lactococcus lactis (pyc). Other pathways to pyruvate and oxaloacetate are known to those skilled in the art and may be applied to supply these intermediates for the indicated biosynthetic pathways.

Also, as depicted in FIG. 1, the enzyme formate lyase, such as from E. coli (pflB), may catalyze pyruvate to acetyl-CoA and formate (the consumption of one CoA not shown in FIG. 1). Formate dehydrogenase, such as from E. coli (fdoGHI, fdnGHI) then catalyzes the oxidation of formate to carbon dioxide, with two electrons reducing menaquinol (see second sheet of FIG. 1). Reduced menaquinol, shown as MQH₂, may then provide reducing equivalents to a subsequent menaquinol-dependent fumarate reductase, such as of E. coli (frdABCD), discussed above for biosynthetic pathway C.

Expression or activity of these genes/enzymes can be changed or evolved to any desired environment, including aerobic, anaerobic, and microaerobic.

As noted above, the enzymes noted are exemplary and not meant to be limiting. The level of skill in biotechnological and recombinants arts is high and the knowledge of enzymes is large and ever-expanding, as evidenced by the readily available knowledge that may be found in the art, as exemplified by the information on the following searchable database websites: www.metacyc.org; www.ecocyc.org; and www.brenda-enzymes.info. One skilled in the art is capable with limited research and routine experimentation to identify any number of genetic sequences either experimentally via directed screening or the assessment of libraries or from sequence databases that encode the desired enzymatic functions. One skilled in the art would then with routine experimentation be able to express these enzymatic functions in a desired recombinant host.

The following summarizes the overall mass balance of sugar-based biosynthetic pathways A, B and C of FIG. 1. Production from glucose: 1 glucose ->1 1,4-BDO+2 CO₂+2 protons. A combination of pathways A, B and C may be used simultaneously in a recombinant host to achieve this mass balance as well as an electron balance.

It is within the presently conceived scope of the invention, at least for some embodiments, to genetically modify a microorganism of interest to comprise both 1) introduced genetic elements (i.e., heterologous nucleotide sequences) providing enzymatic function to complete one of the 1,4-BDO biosynthetic pathways described herein and 2) introduced genetic elements (i.e., heterologous nucleotide sequences) providing enzymatic function(s) directed to increasing the microorganism's tolerance to 1,4-BDO. Improvement of tolerance to 1,4-BDO by a recombinant 1,4-BDO-synthesizing microorganism is considered important in order to achieve more cost-effective industrial systems for 1,4-BDO biosynthesis. This is related at least in part to higher downstream separation costs when 1,4-BDO final titers are relatively low at the end of an industrial system biosynthetic process.

In the following examples, efforts have been made to ensure accuracy with respect to numbers used (e.g., amounts, temperatures, etc.), but some experimental error and deviation should be accounted for. Unless indicated otherwise, temperature is in degrees Celsius and pressure is at or near atmospheric pressure at approximately 5340 feet (1628 meters) above sea level. It is noted that work done at external analytical and synthetic facilities was not conducted at or near atmospheric pressure at approximately 5340 feet (1628 meters) above sea level. All reagents, unless otherwise indicated, were obtained commercially.

The meaning of abbreviations is as follows: “C” means Celsius or degrees Celsius, as is clear from its usage, “s” means second(s), “min” means minute(s), “h” means hour(s), “psi” means pounds per square inch, “nm” means nanometers, “d” means day(s), “μL” means microliter(s), “mL” means milliliter(s), “L” means liter(s), “mm” means millimeter(s), “nm” means nanometers, “mM” means millimolar, “mM” means micromolar, “M” means molar, “mmol” means millimole(s), “mmol” means micromole(s)”, “g” means gram(s), “mg” means microgram(s) and “μg” means nanogram(s), “PCR” means polymerase chain reaction, “OD” means optical density, “OD₆₀₀” means the optical density measured at a wavelength of 600 nm, “kDa” means kilodaltons, “g” means the gravitation constant, “bp” means base pair(s), “kbp” means kilobase pair(s), “% w/v” means weight/volume percent, % v/v″ means volume/volume percent, “IPTG” means isopropyl-D-thiogalactopyranoiside, “RBS” means ribosome binding site, “HPLC” means high performance liquid chromatography, and “GC” means gas chromatography.

### EXAMPLES SECTION

Sequences described in the following examples are disclosed in the sequence listing at the end of this application.

Microbial Hosts for 1,4-BDO Bio-Production, General Discussion

Microbial hosts for 1,4-BDO bio-production may be selected from bacteria, cyanobacteria, filamentous fungi and yeasts. The microbial host used for 1,4-BDO bio-production may have a degree of inherent tolerance to 1,4-BDO so that some yield is not limited by 1,4-BDO toxicity. However, microbes that are metabolically active at high titer levels of 1,4-BDO are not yet well known in the art.

The microbial hosts selected for the production of 1,4-BDO may have a degree of inherent tolerance to 1,4-BDO and may also be able to convert carbohydrates to 1,4-BDO at some level. The criteria for selection and/or ongoing evaluations of suitable microbial hosts include the following: at least some intrinsic tolerance to 1,4-BDO, high rate of glucose utilization, high rates and yields of conversion of sugar substrates to 1,4 BDO (after introduction of genetic elements such as provided herein) and the availability of genetic tools for gene manipulation, and the ability to generate stable chromosomal alterations.

Suitable host strains with a tolerance for 1,4-BDO may be identified initially by screening based on the intrinsic tolerance of the strain. The intrinsic tolerance of microbes to 1,4-BDO may be measured by determining the (MIC) or minimum inhibitory concentration of 1,4-BDO that is responsible for complete inhibition of growth in a given environment and media. The MIC values may be determined using methods known in the art. In addition several other methods of determining microbial tolerance may be used, not limited to but including, minimum bacteriocidal concentration (MBC), which is the minimum concentration needed to completely kill all cells in a microbial culture in a given environment and media. Alternatively, one may determine the IC50, which is the concentration of 1,4 BDO that is responsible for 50% inhibition of the growth rate when grown in a defined media and environment. The MIC, MBC and IC50 values may be determined using methods known in the art. For example, the microbes of interest may be grown in the presence of various amounts of 1,4-BDO and the growth rate monitored by measuring the optical density at 600 nanometers. The doubling time may be calculated from the logarithmic part of the growth curve and used as a measure of the growth rate.

Microbial hosts initially selected for 1,4-BDO bio-production should also utilize sugars including glucose at a high rate. Most microbes are capable of utilizing carbohydrates. However, certain environmental microbes cannot utilize carbohydrates to high efficiency, and therefore would not be suitable hosts.

The ability to genetically modify the host is essential for the production of any recombinant microorganism. The mode of gene transfer technology may be by electroporation, conjugation, transduction or natural transformation. A broad range of host conjugative plasmids and drug resistance markers are available. The cloning vectors are tailored to the host organisms based on the nature of antibiotic resistance markers that can function in that host.

The microbial host may also be manipulated in order to inactivate competing pathways for carbon flow by deleting various genes. This may require the availability of either transposons to direct inactivation or chromosomal integration vectors. Additionally, the bio-production host may be amenable to chemical mutagenesis so that mutations to improve intrinsic 1,4-BDO tolerance may be obtained.

Based on the various criteria described above, suitable microbial hosts for the production of 1,4-BDO generally may include, but are not limited to, any gram negative organisms such as E. coli, or Pseudomononas sp.; any gram positive microorganism, for example Bacillus subtilis, Lactobaccilus sp. or Lactococcus sp. a yeast, for example Saccharomyces cerevisiae, Pichia pastoris or Pichia stipitis; and other groups or microbial species. More particularly, suitable microbial hosts for the production of 1,4-BDO generally include, but are not limited to, members of the genera Clostridium, Zymomonas, Escherichia, Salmonella, Rhodococcus, Pseudomonas, Bacillus, Lactobacillus, Enterococcus, Alcaligenes, Klebsiella, Paenibacillus, Arthrobacter, Corynebacterium, Brevibacterium, Pichia, Candida, Hansenula and Saccharomyces. Hosts that may be particularly of interest include: Escherichia coli, Alcaligenes eutrophus, Bacillus licheniformis, Paenibacillus macerans, Rhodococcus erythropolis, Pseudomonas putida, Lactobacillus plantarum, Enterococcus faecium, Enterococcus gallinarium, Enterococcus faecalis, Bacillus subtilis and Saccharomyces cerevisiae.

In view of the above disclosure, the following pertain to exemplary methods of modifying specific species of host organisms that span a broad range of microorganisms of commercial value. As noted, the use of E. coli, although convenient for many reasons, is not meant to be limiting.

### 1,4-BDO Specific, Non-Limiting Technical Examples

The following examples disclose specific methods for providing an E. coli cell with heterologous nucleic acid sequences that encode for enzymes required for synthesis of 1,4-BDO in E. coli according to any biosynthetic pathways A, B and C. Where there is a method to achieve a certain result that is commonly practiced in two or more specific examples, that method may be provided in a separate Common Methods section that follows the examples. Each such common method is incorporated by reference into the respective specific example that so refers to it. Also, where supplier information is not complete in a particular example, additional manufacturer information may be found in a separate Summary of Suppliers section that may also include product code, catalog number, or other information. This information is intended to be incorporated in respective specific examples that refer to such supplier and/or product.

### Example 1

### Cloning of M. tuberculosis kgd (for Pathway A)

A nucleic acid sequence encoding the protein sequence for the alpha-ketoglutarate decarboxylase from M. tuberculosis (kgd) was codon optimized for enhanced protein expression in E. coli according to a service from DNA 2.0 (Menlo Park, Calif. USA), a commercial DNA gene synthesis provider. This thus-codon-optimized nucleic acid sequence incorporated an NcoI restriction site overlapping the gene start codon and was followed by a HindIII restriction site. In addition a Shine Delgarno sequence or ribosomal binding site was placed in front of the start codon preceded by an EcorI restriction site. This nucleic acid sequence (SEQ ID NO:0001) was synthesized by DNA 2.0 and provided in a pJ206 vector backbone.

### Example 2

### Cloning of C. kluvveri 4hbd (common for Pathways A, B & C)

C. kluyveri DSMZ # 555 was obtained from the German Collection of Microorganisms and Cell Cultures (Braunschweig, Germany) (“DSMZ”) and cultures grown as described in Subsection 1, Bacterial Growth Methods in Common Methods Section, below. Genomic DNA from C. kluyveri cultures was obtained from a Qiagen (Valencia Calif. USA) genomic DNAEasy kit according to manufacturer's instructions. The following oligonucleotides were obtained from the commercial provider Operon. Primer 1: TCTAGAGTATATAAGGAGGAAAAAATATGAAGTTATTAAAATTG (SEQ ID NO: NO:0015) and Primer 2: CCCGGGTTACATATTAATATAACTTTTTATATGTGTTTACTATGT (SEQ ID NO: NO:0016). Primer 1 contains an XbaI restriction site while Primer 2 contains a SmaI restriction site. These primers were used to amplify the 4hbd region from C. kluyveri genomic DNA using standard polymerase chain reaction (PCR) methodologies. The sequence of the resultant PCR product is given in SEQ ID NO:0002. The 4hbd gene region (SEQ ID NO:0002), can be subcloned into any number of commercial cloning vectors including but not limited to pCR2.1-topo (Invitrogen Carlsbad, Calif. USA), other topo-isomerase based cloning vectors (Invitrogen Corp., Carlsbad, Calif. USA) the pSMART-series of cloning vectors from Lucigen (Middleton, Wis. USA) or the Strataclone series of vectors (Stratagene, La Jolla, Calif. USA) after amplification by PCR.

### Example 3

### Cloning of C. braakii dhaT (common for Pathways A, B & C)

C. braakii DSMZ # 30040 was obtained from DSMZ and cultures grown as described in Subsection 1, Bacterial Growth Methods in Common Methods Section, below. Genomic DNA from C. braakii cultures was obtained from a Qiagen (Valencia Calif. USA) genomic DNAEasy kit according to manufacturer's instructions. The following oligonucleotides were obtained from the commercial provider Operon. Primer 1: CCCGGGCTAAGAAGGTATATTATGAGCTATCGTATGTTTG (SEQ ID NO: NO:0017) and Primer 2: GCGGCCGC GCGTTATCAGAATGCCTGACG (SEQ ID NO: NO:0018). Primer 1 contains an SmaI restriction site while Primer 2 contains a NotI restriction site. These primers were used to amplify the dhaT region from C. braakii genomic DNA using standard polymerase chain reaction (PCR) methodologies. The sequence of the resultant PCR product is given in SEQ ID NO:0003. This sequence is subclonable into any number of commercial cloning vectors including but not limited to pCR2.1-topo (Invitrogen Corp., Carlsbad, Calif. USA), other topo-isomerase based cloning vectors (Invitrogen, Carlsbad, Calif. USA) the pSMART-series of cloning vectors from Lucigen (Middleton, Wis. USA) or the Strataclone series of vectors (Stratagene, La Jolla, Calif. USA) after amplification by PCR.

### Example 4

### Cloning of C. acetobutylicum thiL (for Pathway B)

C. acetobutylicum DSMZ # 792/ATCC #824 was obtained from DSMZ and cultures grown as described in Bacterial Growth Methods in Common Methods Section, below. Genomic DNA from C. acetobutylicum cultures was obtained from a Qiagen (Valencia Calif. USA) genomic DNAEasy kit according to manufacturer's instructions. The following oligonucleotides were obtained from the commercial provider Operon. Primer 1: GAATTCGGAGGAGTAAAACATGAGAGATGT AGTAAT (SEQ ID NO:0019) and Primer 2: AAGCTTAGTCTCTTTCAACTACGA (SEQ ID NO:0020). Primer 1 contains a SmaI restriction site while Primer 2 contains a HindIII restriction site. These primers have been used to amplify the thiL region from C. acetobutylicum genomic DNA using standard polymerase chain reaction (PCR) methodologies (Inui et al, Applied Genetics and Molecular Biotechnology. (2008), 77:1305-1316). The sequence of the resultant PCR product is given in SEQ ID NO: 0004. This sequence is subclonable into any number of commercial cloning vectors including but not limited to pCR2.1-topo (Invitrogen Corp., Carlsbad, Calif. USA), other topo-isomerase based cloning vectors (Invitrogen, Carlsbad, Calif. USA) the pSMART-series of cloning vectors from Lucigen (Middleton, Wis. USA) or the Strataclone series of vectors (Stratagene, La Jolla, Calif. USA) after amplification by PCR.

### Example 5

### Cloning of C. acetobutylicum crt, bcd, etfB, etfA and hbd genes (for use in Pathway B)

C. acetobutylicum DSMZ # 792/ATCC #824 is obtained from DSMZ and cultures is grown as described in Subsection 1, Bacterial Growth Methods in Common Methods Section, below. Genomic DNA from C. acetobutylicum cultures is obtained from a Qiagen (Valencia Calif. USA) genomic DNAEasy kit according to manufacturer's instructions. The following oligonucleotides are obtained from the commercial provider Operon. Primer 1: ATCCCGGGATATTTTAGGAGGATTAGTCATGGAACTAAACAATG (SEQ ID:0021) and Primer 2: ATCCCGGGAGATCTTGTAAACTTA TTTTGAATAA TCGTAGAAACCC (SEQ ID NO:0022). Primer 1 contains a SmaI restriction site while Primer 2 contains both a SmaI and a BglII restriction site. These primers are used to amplify the crt, bcd, etfB, etfA, hbd operon region from C. acetobutylicum genomic DNA using standard polymerase chain reaction (PCR) methodologies. The sequence of the resultant PCR product is given in SEQ ID NO:0005. This sequence is subclonable into any number of commercial cloning vectors including but not limited to pCR2.1-topo (Invitrogen Corp., Carlsbad, Calif. USA), other topo-isomerase based cloning vectors (Invitrogen, Carlsbad, Calif. USA) the pSMART-series of cloning vectors from Lucigen (Middleton, Wis. USA) or the Strataclone series of vectors (Stratagene, La Jolla, Calif. USA) after amplification by PCR.

### Example 6

### Cloning of C. acetobutylicum crt-hbd genes (for Pathway B)

The crt, bcd, etfB, etfA, hbd operon (SEQ ID NO:0005) from Example 5, is subcloned into any of a number of commercial cloning vectors including but not limited to pCR2.1-topo (Invitrogen Corp., Carlsbad, Calif. USA), other topo-isomerase based cloning vectors (Invitrogen, Carlsbad, Calif. USA) the pSMART-series of cloning vectors from Lucigen (Middleton, Wis. USA) or the Strataclone series of vectors. (Stratagene, La Jolla, Calif. USA) after amplification by PCR. After this subcloning step, routine methods known in the art may be used to remove the internal DNA sequence corresponding to the bcd, etfB and etfA genes to generate an operon containing only the crt and hbd genes. One example is to perform another PCR amplification on the complete circular cloning vector containing the crt, bcd, etfB, etfA, hbd operon (SEQ ID NO:0005) with the following two primers Primer1: GCATTGATAGTTTCTTTAAATTTAGGGAGG (SEQ ID NO: NO:0023) and Primer2: CTCCTATCTATTTTTGAAGCCTTCAATTTTTC(SEQ ID NO: NO:0024). This will result in a linear fragment of DNA that when treated with polynucleotide kinase, ligated with T4 ligase and electroporated into E. coli will result in a subcloned DNA sequence containing only the crt and hbd genes flanked by a SmaI restriction site on the 5′ end and both a SmaI and BglII restrictipon site on the 3′ end (SEQ ID NO:0006).

### Example 7

### Cloning of C. aminobutyricum abfD and abfT genes (for Pathway B)

C. aminobutyricum DSMZ# 2634 is obtained from DSMZ and cultures grown as described in Subsection I, Bacterial Growth Methods in Common Methods Section, below. Genomic DNA from C. aminobutyricum cultures is obtained from a Qiagen (Valencia Calif. USA) genomic DNAEasy kit according to manufacturer's instructions. The following oligonucleotides are obtained from the commercial provider Operon. Primer 1: GTTTAAA CATT ATTTTAAGAA GGAGTGATTA TATTATGTTA (SEQ ID NO:0025) and Primer 2: CCCGGG CGA TCTGGTTCCA ATTAGAATGC CGCGTTGAAT (SEQ ID NO:0026), Primer 1 contains a PmeI restriction site while Primer 2 contains a SmaI restriction site. These primers are used to amplify the abfDT region from C. aminobutyricum genomic DNA using standard polymerase chain reaction (PCR) methodologies. The sequence of the resultant PCR product is given in SEQ ID NO:0007. This sequence is subclonable into any number of commercial cloning vectors including but not limited to pCR2.1-topo (Invitrogen Corp., Carlsbad, Calif. USA), other topo-isomerase based cloning vectors (Invitrogen, Carlsbad, Calif. USA) the pSMART-series of cloning vectors from Lucigen (Middleton, Wis. USA) or the Strataclone series of vectors (Stratagene, La Jolla, Calif. USA) after amplification by PCR.

### Example 8

### Construction of Cloning Vector pKK223-MCS1

A circular plasmid based cloning vector termed pKK223-MCS1 for expression of genes for 1,4 BDO biosynthesis in E. coli was constructed as follows. An E. coli cloning strain bearing pKK223-aroH was obtained as a kind a gift from the laboratory of Prof. Ryan T. Gill from the University of Colorado, Boulder. Cultures of an E. coli cloning strain bearing the plasmid were grown by standard methodologies (see Subsection II, Common Methods Section, below), and plasmid DNA was prepared by a commercial miniprep column from Qiagen (Valencia Calif. USA). Plasmid DNA was digested with the restriction endonucleases EcorI and HindIII obtained from New England BioLabs (Ipswitch, Mass. USA) according to manufacturer's instructions. This digestion served to separate the aroH reading frame from the pKK223 backbone. The digestion mixture was separated by agarose gel electrophoresis, and visualized under UV transillumination as described in the Common Methods Section, subsection II, below. An agarose gel slice containing a DNA piece corresponding to the backbone of the pKK223 plasmid was cut from the gel and the DNA recovered with a standard gel extraction protocol and components (Cat. No. 28706) from Qiagen (Valencia Calif. USA) according to manufacturer's instructions.

The following oligonucleotides were obtained from the commercial provider Operon (Huntsville, Ala. USA). Oligonucleotide 1: [Phos]AATTCGCAT TAAGCTTGCA CTCGAGCGTC GACCGTTCTA GACGCGATATCCGAATCCCG GGCTTCGTGC GGCCGC (SEQ ID NO: 0027) and Oligonucleotide 2: [Phos]AGCTGCGGCC GCACGAAGCC CGGGATTCGG ATATCGCGTC TAGAACGGTC GACGCTCGAG TGCAAGCTTA ATGCG (SEQ ID NO:28). [Phos] indicates a 5′ phosphate. These oligonucleotides were mixed in a 1:1 ratio 50 micromolar concentration in a volume of 50 microliters and hybridized in a thermocycler with the following temperature cycles. 95 C for 10 minutes, 90 C for 5 minutes, 85 C for 10 minutes, 80 C for 5 minutes, 75 C for 5 minutes, 70 C for 1 minutes, 65 C for 1 minutes, 55 C for 1 minutes, and then cooled to 4 C. This double stranded piece of DNA, comprising multiple cloning sites, has 5′ overhangs corresponding to overhangs of EcorI and HindIII restriction sites. This piece was diluted in Deionized water 1:100 and ligated according to and with components of the Ultraclone Cloning Kit (Lucigen Middleton, Wis. USA) into the gel extracted EcorI, HindIII digested pKK223 backbone. The ligation product was transformed and electroporated according to manufacturer's instructions. The predicted sequence of the resulting vector termed pKK223-MCS1 (SEQ ID NO:0008) was confirmed by routine sequencing performed by the commercial service provided by Macrogen (Rockville, Md. USA). pKK223-MCS1 confers resistance to beta-lactamase and contains a new multiple cloning site and a ptac promoter inducible in E. coli hosts by IPTG.

### Example 9

### Construction of Cloning Vector pKK223-MCS2

A circular plasmid based cloning vector termed pKK223-MCS2 for expression of genes for 1,4 BDO synthesis in E. coli was constructed as follows. An E. coli 10G F′ cloning strain (Lucigen, Madison Wis.) bearing pKK223-MCS1 was obtained from Example 8. Cultures of an E. coli cloning strain bearing the plasmid were grown by standard methodologies (see Subsection II, Common Methods Section, below), and plasmid DNA was prepared by a commercial miniprep column from Qiagen (Valencia Calif. USA). Plasmid DNA was digested with the restriction endonuclease XbaI and treated with antarctic phosphatase, both enzymes were obtained from New England BioLabs (Ipswitch, Mass. USA) and reactions are carried out according to manufacturer's instructions. This digestion served to linearize the vector backbone. The digestion mixture was separated by agarose gel electrophoresis, and visualized under UV transillumination as described in the Common Methods Section, subsection II, below. An agarose gel slice containing a DNA piece corresponding to the backbone of the linear vector was cut from the gel and the DNA recovered with a standard gel extraction protocol and components from Qiagen (Valencia Calif. USA) according to manufacturer's instructions.

The following oligonucleotides were obtained from the commercial provider Operon. Oligonucleotide 1: CTAG TTTAAA CATATTCTGA AATGAGCTGT TGACAATTAA TCATCGGCTC GTATAATGTG (SEQ ID NO:0029), Oligonucleotide 2: [Phos] TGGAATTGTG AGCGGATAAC AATTTCACAC ACAT (SEQ ID NO:0030, Oligonucleotide 3: CTAGATGTGTGTGAAATTGT TATCCGCTCA CAATTCCACA CATTATACGAGCCGATGA (SEQ ID NO:0031) and Oligo4: [Phos] TTAATTGTCA ACAGCTCATT TCAGAATATG TTTAAA (SEQ ID NO:0032). [Phos] indicates a 5′ phosphate. These oligonucleotides were mixed in a 1:1 ratio 50 micromolar concentration in a volume of 50 microliters and hybridized to form a double stranded piece of DNA in a thermocycler with the following temperature cycles. 95 C for 10 minutes, 90 C for 5 minutes, 85 C for 10 minutes, 80 C for 5 minutes, 75 C for 5 minutes, 70 C for 5 minutes, 65 C for 5 minutes, 60 C for 5 minutes, 55 C for 10 minutes, 50 C for 10 minutes, 45 C for 5 minutes, 40 C for 5 minutes, and then cooled to 4 C. This double stranded piece of DNA, comprising multiple cloning sites, has 5′ overhangs corresponding to overhangs of an XbaI restriction sites. This piece is diluted in Deionized water 1:100 and ligated according to and with components of the Ultraclone Cloning Kit (Lucigen Middleton, Wis. USA) into the gel extracted XbaI digested and antarctic phosphatase treated pKK223-MCS1. The ligation product is transformed and electroporated according to manufacturer's instructions. The predicted sequence of the resulting vector termed pKK223-MCS1 (SEQ ID NO:0009) is confirmed by routine sequencing performed by the commercial service provided by Macrogen (Rockville, Md. USA). pKK223-MCS2 confers resistance to beta-lactamase and contains 2 ptac promoters inducible in E. coli hosts by IPTG associated with 2 multiple cloning sites.

### Example 10

### Construction of 1,4-BDO production plasmid pBDO-1

To co-express the genes in the 1,4-BDO biosynthetic pathway A needed for the supplementary enzymatic functions necessary for the production of 1,4-BDO in E. coli, the production plasmid pBDO-1 is constructed as follows. All restriction endonucleases and antarctic phosphatase are obtained from New England BioLabs (Ipswitch, Mass. USA) and all reactions are carried out according to manufacturer's instructions. Cultures of an E. coli cloning strain bearing subclones are cultured by standard methodologies (see Subsection II, Common Methods Section, below), and all plasmid DNA is prepared by a commercial miniprep column from Qiagen (Valencia Calif. USA). The digestion mixtures are separated by routine agarose gel electrophoresis, and visualized under UV transillumination as described in the Common Methods Section, subsection II, below. Agarose gel slices containing desired DNA pieces are cut from the gel and the DNA recovered with a standard gel extraction protocol and components from Qiagen (Valencia Calif. USA) according to manufacturer's instructions. Ligations and transformations are also carried out as described in the Common Methods Section, subsection II, below. EcorI, HindIII digested and antarctic phosphatase treated pKK223-MCS1 plasmid is first ligated with the DNA sequence containing the kgd gene (SEQ ID NO:0001) which has been prepared by an EcorI and HindIII digest. After ligation and transformation, a new plasmid termed pKK223-MCS1-kgd is obtained. XbaI, SmaI digested and antarctic phosphatase treated pKK223-MCS1-kgd plasmid is then ligated with the DNA sequence containing the 4hbd gene (SEQ ID NO:0002) which has been prepared by an XbaI and SmaI digest. After ligation and transformation, a new plasmid termed pKK223-MCS1-kgd-4-hbd is obtained. SmaI, NotI digested and antarctic phosphatase treated pKK223-MCS1-kgd-4-hbd plasmid is then ligated with the DNA sequence containing the dhaT gene (SEQ ID NO:0003) which has been prepared by an SmaI and NotI digest. After ligation and transformation, a new plasmid termed pBDO-1 is obtained (SEQ ID NO:0010). This example is not the only embodiment envisioned of this pathway which may be practiced in numerous hosts under expression of numerous promoters on vectors or integrated into the host chromosome.

### Example 11

### Construction of 1,4-BDO synthesis plasmid pBDO-2

To co-express the genes in the 1,4-BDO biosynthetic pathway B needed for the supplementary enzymatic functions necessary for the production of 1,4-BDO in E. coli, the production plasmid pBDO-2 is constructed as follows. All restriction endonucleases and antarctic phosphatase are obtained from New England BioLabs (Ipswitch, Mass. USA) and all reactions are carried out according to manufacturer's instructions. Cultures of an E. coli cloning strains bearing subclones are cultured by standard methodologies (see Subsection II, Common Methods Section, below), and all plasmid DNA is prepared by a commercial miniprep column from Qiagen (Valencia Calif. USA). The digestion mixtures are separated by routine agarose gel electrophoresis, and visualized under UV transillumination as described in the Common Methods Section, subsection II, below. Agarose gel slices containing desired DNA pieces are cut from the gel and the DNA recovered with a standard gel extraction protocol and components from Qiagen (Valencia Calif. USA) according to manufacturer's instructions. Ligations and transformations are also carried out as described in the Common Methods Section, subsection II, below.

EcorI, HindIII digested and antarctic phosphatase treated pKK223-MCS2 plasmid is first ligated with the DNA sequence containing the thiL gene (SEQ ID NO:0004) which has been prepared by an EcorI and HindIII digest. After ligation and transformation, a new plasmid termed pKK223-MCS2-thiL is obtained. PmeI digested and antarctic phosphatase treated pKK223-MCS2-thiL plasmid is then ligated with the DNA sequence containing the crt-hbd gene (SEQ ID NO: 0006) which has been prepared by an SmaI digest. After ligation and transformation, a new plasmid termed pKK223-MCS2-thil-crt-hbd is obtained. SmaI digested and antarctic phosphatase treated pKK223-MCS2-thil-crt-hbd plasmid is then ligated with the DNA sequence containing the abfD and abfT genes (SEQ ID NO:0007) which has been prepared by a PmeI and SmaI digest. After ligation and transformation, a new plasmid termed pKK223-MCS2-thil-crt-hbd-abfDT is obtained. SmaI, NotI digested and antarctic phosphatase treated pKK223-MCS2-thil-crt-hbd-abfDT plasmid is then ligated with the DNA sequence containing the dhaT gene (SEQ ID NO:0003) which has been prepared by an SmaI and NotI digest. After ligation and transformation, a new plasmid termed pBDO-2 is obtained (SEQ ID NO:0011). This example is not the only embodiment envisioned of this pathway which may be practiced in numerous host under expression of numerous promoters on vectors or integrated into the host chromosome.

### Example 12

### Construction of 1,4-BDO synthesis strain 1: E. coli JW1375+pBDO-1

pBDO-1 is prepared and transformed as provided herein into E. coli JW1375, which is an E. coli with a deletion of the ldhA gene obtained as part of the Keio E. coli Gene Deletion Collection from the commercial provider Open Biosystems (Huntsville, Ala. USA). The resulting clone E. coli JW1375+pBDO-1 is cultured under anaerobic conditions under induction with 1 mM IPTG and the supernatant assessed for the presence of 1,4-BDO according to standard procedures described in the Common Methods Section, subsection III, below.

1,4-BDO is obtained in a measurable quantity at the conclusion of a bio-production event (see types of bio-production events, below, incorporated by reference into this Example). That measurable quantity is substantially greater than a quantity of 1,4-BDO produced in a control bio-production event of a control selected from: E. coli JW1375 lacking transformation with pBDO-1; E. coli JW1375 transformed with a plasmid similar to pBDO-1 but lacking functional nucleic acid sequences provided in the latter; and other suitable control organism.

### Example 13

### Construction of 1,4-BDO synthesis strain 2: E. coli JW1375+pBDO-2

pBDO-2 is prepared and transformed as provided herein into E. coli JW1375, which is an E. coli with a deletion of the ldhA gene obtained as part of the Keio E. coli Gene Deletion Collection from the commercial provider Open Biosystems. The resulting clone E. coli JW1375+pBDO-2 is cultured under anaerobic conditions under induction with 1 mM IPTG and the supernatant assessed for the presence of 1,4-BDO according to standard procedures described in the Common Methods Section, subsection III, below.

1,4-BDO is obtained in a measurable quantity at the conclusion of a bio-production event (see types of bio-production events, below, incorporated by reference into this Example). That measurable quantity is substantially greater than a quantity of 1,4-BDO produced in a control bio-production event of a control selected from: E. coli JW1375 lacking transformation with pBDO-2; E. coli JW1375 transformed with a plasmid similar to pBDO-2 but lacking functional nucleic acid sequences provided in the latter; and other suitable control organism.

### Example 14

### Cloning of E. coli yneI Gene

E. coli K12 is obtained from the Yale Genetic Stock Center (New Haven, Conn.) and cultures are grown as described in Methods. Genomic DNA from E. coli K12 cultures is obtained from a Qiagen genomic DNAEasy kit according to manufacturer's instructions.

The following oligonucleotides are obtained from the commercial provider Operon. Primer 1: TCTAGAAGAGTAAATC TGCGTATCTT CATACCATGA (SEQ ID NO:0033) and Primer 2: CTCGAGTCAGATCCGG TCTTTCCACA CCGTCTGGAT (SEQ ID NO:0034) Primer 1 contains an XbaI restriction site while Primer 2 contains a XhoI restriction site. These primers are used to amplify the yneI region from E. coli K12 genomic DNA using standard polymerase chain reaction (PCR) methodologies. The predicted sequence of the resultant PCR product is given in SEQ ID NO:0012. The amplified PCR product is separated by routine agarose gel electrophoresis, and is visualized under UV transillumination as described in the Common Methods Section, subsection II, below. An agarose gel slice containing the desired DNA piece is cut from the gel and the DNA is recovered with a standard gel extraction protocol and components from Qiagen according to manufacturer's instructions. The purified yneI PCR product is ligated into the pCR2.1-topo-TA cloning vector and transformed into a Top10F E. coli host strain from Invitrogen (Carlsbad, Calif.) according to manufacturer's instructions. DNA sequence is confirmed by routine sequencing services provided by Macrogen (USA).

### Example 15

### Construction of 1,4-BDO Production Plasmid pBDO-3

To co-express the genes in the 1,4-BDO biosynthetic pathway C needed for the enzymatic functions necessary for the production of 1,4-BDO, the production plasmid pBDO-1 is constructed as follows. All restriction endonucleases and antarctic phosphatase are obtained from New England BioLabs and all reactions are carried out according to manufacturer's instructions. Cultures of an E. coli cloning strain bearing subclones are cultured using standard methodologies and all plasmid DNA is prepared by a commercial miniprep column from Qiagen (Valencia Calif. USA). The digestion mixtures are separated by routine agarose gel electrophoresis, and are visualized under UV transillumination as described in the Common Methods Section, subsection II, below. Agarose gel slices containing desired DNA pieces are cut from the gel and the DNA recovered with a standard gel extraction protocol and components from Qiagen according to manufacturer's instructions. Ligations and transformations are also carried out as described in the Common Methods Section, subsection II, below.

HindIII, XhoI digested and antarctic phosphatase treated pKK223-MCS1 plasmid is first ligated with the DNA sequence containing the yneI gene (SEQ ID NO:0012) which is prepared from the backbone vector pCR2.1-topo-yneI (SEQ ID NO:0013) by an HindIII and XhoI digest. After ligation and transformation, a new plasmid termed pKK223-MCS1-yneI is obtained. XhoI, NotI digested and antarctic phosphatase treated pKK223-MCS1-yneI plasmid is then ligated with the DNA sequence containing the 4hbd and dhaT nucleic acid sequences, which is prepared by an XhoI and NotI digest of pBDO-1 (see Examples 2, 3 and 10, incorporated by reference into this Example). After ligation and transformation, a new plasmid termed pBDO-3 is obtained (SEQ ID NO:0014). This example is not the only embodiment envisioned for this pathway which may be practiced in numerous host organisms under expression of numerous promoters on vectors or integrated into the host chromosome.

### Example 16

### Construction of 1,4-BDO Synthesis Strain 3: E. coli NZN111+pBDO-3

pBDO-3 is prepared and is transformed into E. coli NZN111, which is a succinate producing strain of E. coli with mutations in both the ldhA and pflB genes obtained from the E. coli genetic stock Center (New haven, CT). The resulting clone E. coli NZN111+pBDO-3 is cultured under anaerobic conditions under induction with 1 mM IPTG and the supernatant assessed for the presence of 1,4-BDO according to standard procedures described in Subsection III of Common Methods Section, below.

Further, using such methods 1,4-BDO is obtained in a measurable quantity at the conclusion of a bio-production event (see types of bio-production events, below, incorporated by reference into this Example). That measurable quantity is substantially greater than a quantity of 1,4-BDO produced in a control bio-production event of a control selected from: E. coli NZN111 lacking transformation with pBDO-3; E. coli NZN 111 transformed with a plasmid similar to pBDO-3 but lacking functional nucleic acid sequences provided in the latter; and other suitable control organism.

Examples 10-16 add supplementary enzymes to an E. coli to compete a desired biosynthetic pathway for production of 1,4-BDO. However, given the high level of skill in the art, in combination with the present disclosure, other species may be genetically engineered to obtain recombinant microorganisms that produce 1,4-BDO. More or less enzyme-encoding nucleotide sequences than were added in the above examples may need to be added in for a particular species. However, it is within the scope of the present invention to so practice the invention in species other than E. coli, inserting heterologous nucleotide sequences as needed to provide a functional 1,4-BDO biosynthetic pathway, supplementing existing enzymes where available and appropriate. The following are non-limiting general examples directed to practicing the present invention in other microorganism species.

### General Example 17

### Expression of an 1,4-BDO Biosynthetic Pathway in Rhodococcus erythropolis

A series of E. coli-Rhodococcus shuttle vectors are available for expression in R. erythropolis, including, but not limited to, pRhBR17 and pDA71 (Kostichka et al., Appl. Microbiol. Biotechnol. 62:61-68 (2003)). Additionally, a series of promoters are available for heterologous gene expression in R. erythropolis (see for example Nakashima et al., Appl. Environ. Microbiol. 70:5557-5568 (2004), and Tao et al., Appl. Microbiol. Biotechnol. 2005, DOI 10.1007/s00253-005-0064). Targeted gene disruption of chromosomal genes in R. erythropolis may be created using the method described by Tao et al., supra, and Brans et al. (Appl. Environ. Microbiol. 66: 2029-2036 (2000)). These published resources are incorporated by reference for their respective indicated teachings and compositions.

The heterologous genes required for the production of 1,4-BDO, as described above, may be cloned initially in pDA71 or pRhBR71 and transformed into E. coli. The vectors may then be transformed into R. erythropolis by electroporation, as described by Kostichka et al., supra. The recombinants may be grown in synthetic medium containing glucose and the production of 1,4-BDO can be followed using methods known in the art.

### General Example 18

### Expression of an 1,4-BDO Biosynthetic Pathway in B. subtilis

Methods for gene expression and creation of mutations in B. subtilis are also well known in the art. For example, the genes of an 1,4-BDO biosynthetic pathway may be isolated from various sources, cloned into a modified vector and transformed into Bacillus subtilis strains.

### General Example 19

### Expression of an 1,4-BDO Biosynthetic Pathway in B. licheniformis

Most of the plasmids and shuttle vectors that replicate in B. subtilis may be used to transform B. licheniformis by either protoplast transformation or electroporation. The genes required for the production of 1,4-BDO may be cloned in plasmids pBE20 or pBE60 derivatives (Nagarajan et al., Gene 114:121-126 (1992)). Methods to transform B. licheniformis are known in the art (for example see Fleming et al. Appl. Environ. Microbiol., 61(11):3775-3780 (1995)). These published resources are incorporated by reference for their respective indicated teachings and compositions.

The plasmids constructed for expression in B. subtilis may be transformed into B. licheniformis to produce a recombinant microbial host that produces 1,4-BDO.

### General Example 20

### Expression of an 1,4-BDO Biosynthetic Pathway in Paenibacillus macerans

Plasmids may be constructed as described above for expression in B. subtilis and used to transform Paenibacillus macerans by protoplast transformation to produce a recombinant microbial host that produces 1,4-BDO.

### General Example 21

### Expression of the 1,4-BDO Biosynthetic Pathway in Alcaligenes (Ralstonia) Eutrophus

Methods for gene expression and creation of mutations in Alcaligenes eutrophus are known in the art (see for example Taghavi et al., Appl. Environ. Microbiol., 60(10):3585-3591 (1994)). This published resource is incorporated by reference for its indicated teachings and compositions. The genes for an 1,4-BDO biosynthetic pathway may be cloned in any of the broad host range vectors described above, and electroporated to generate recombinants that produce 1,4-BDO. The poly(hydroxybutyrate) pathway in Alcaligenes has been described in detail, a variety of genetic techniques to modify the Alcaligenes eutrophus genome is known, and those tools can be applied for engineering an 1,4-BDO biosynthetic pathway.

### General Example 22

### Expression of an 1,4-BDO Biosynthetic Pathway in Pseudomonas putida

Methods for gene expression in Pseudomonas putida are known in the art (see for example Ben-Bassat et al., U.S. Pat. No. 6,586,229, which is incorporated herein by reference for these teachings). The 1,4-BDO pathway genes may be inserted into pUCP18 and this ligated DNA may be electroporated into electrocompetent Pseudomonas putida KT2440 cells to generate recombinants that produce 1,4-BDO.

### General Example 23

### Expression of an 1,4-BDO Biosynthetic Pathway in Saccharomyces cerevisiae

Methods for gene expression in Saccharomyces cerevisiae are known in the art (see for example Methods in Enzymology, Volume 194, Guide to Yeast Genetics and Molecular and Cell Biology (Part A, 2004, Christine Guthrie and Gerald R. Fink (Eds.), Elsevier Academic Press, San Diego, Calif.). This published resource is incorporated by reference for its indicated teachings and compositions. Expression of genes in yeast typically requires a promoter, followed by the gene of interest, and a transcriptional terminator. A number of yeast promoters can be used in constructing expression cassettes for genes encoding an 1,4-BDO biosynthetic pathway, including, but not limited to constitutive promoters FBA, GPD, ADH1, and GPM, and the inducible promoters GAL1, GAL10, and CUP1. Suitable transcriptional terminators include, but are not limited to FBAt, GPDt, GPMt, ERG10t, GAL1t, CYC1, and ADH1. For example, suitable promoters, transcriptional terminators, and the genes of an 1,4-BDO biosynthetic pathway may be cloned into E. coli-yeast shuttle vectors known in the art.

### General Example 24

### Expression of an 1,4-BDO Biosynthetic Pathway in Lactobacillus plantarum

The Lactobacillus genus belongs to the Lactobacillales family and many plasmids and vectors used in the transformation of Bacillus subtilis and Streptococcus may be used for lactobacillus. Non-limiting examples of suitable vectors include pAM.beta.1 and derivatives thereof (Renault et al., Gene 183:175-182 (1996); and O'Sullivan et al., Gene 137:227-231 (1993)); pMBB1 and pHW800, a derivative of pMBB1 (Wyckoff et al. Appl. Environ. Microbiol 62:1481-1486 (1996)); pMG1, a conjugative plasmid (Tanimoto et al., J. Bacteriol. 184:5800-5804 (2002)); pNZ9520 (Kleerebezem et al., Appl. Environ. Microbiol. 63:4581-4584 (1997)); pAM401 (Fujimoto et al., Appl. Environ. Microbiol. 67:1262-1267 (2001)); and pAT392 (Arthur et al., Antimicrob. Agents Chemother. 38:1899-1903 (1994)). Several plasmids from Lactobacillus plantarum have also been reported (e.g., van Kranenburg R, Golic N, Bongers R, Leer R J, de Vos W M, Siezen R J, Kleerebezem M. Appl. Environ. Microbiol. 2005 March; 71(3): 1223-1230).

### General Example 25

### Expression of an 1,4-BDO Biosynthetic Pathway in Enterococcus faecium, Enterococcus gallinarium, and Enterococcus faecalis

The Enterococcus genus belongs to the Lactobacillales family and many plasmids and vectors used in the transformation of Lactobacillus, Bacillus subtilis, and Streptococcus may be used for Enterococcus. Non-limiting examples of suitable vectors include pAM.beta.1 and derivatives thereof (Renault et al., Gene 183:175-182 (1996); and O'Sullivan et al., Gene 137:227-231 (1993)); pMBB1 and pHW800, a derivative of pMBB1 (Wyckoff et al. Appl. Environ. Microbiol. 62:1481-1486 (1996)); pMG1, a conjugative plasmid (Tanimoto et al., J. Bacteriol. 184:5800-5804 (2002)); pNZ9520 (Kleerebezem et al., Appl. Environ. Microbiol. 63:4581-4584 (1997)); pAM401 (Fujimoto et al., Appl. Environ. Microbiol. 67:1262-1267 (2001)); and pAT392 (Arthur et al., Antimicrob. Agents Chemother. 38:1899-1903 (1994)). Expression vectors for E. faecalis using the nisA gene from Lactococcus may also be used (Eichenbaum et al., Appl. Environ. Microbiol. 64:2763-2769 (1998). Additionally, vectors for gene replacement in the E. faecium chromosome may be used (Nallaapareddy et al., Appl. Environ. Microbiol. 72:334-345 (2006)).

For each of the General Examples 17-25, the following 1,4-BDO production comparison may be incorporated thereto: Using analytical methods for 1,4-BDO such as are described in Subsection III of Common Methods Section, below, 1,4-BDO is obtained in a measurable quantity at the conclusion of a respective bio-production event conducted with the respective recombinant microorganism (see types of bio-production events, below, incorporated by reference into each respective General Example). That measurable quantity is substantially greater than a quantity of 1,4-BDO produced in a control bio-production event using a suitable respective control microorganism lacking the functional 1,4-BDO pathway so provided in the respective General Example.

### Common Methods Section

All methods in this Section are provided for incorporation into the above methods where so referenced therein.

Subsection I. Bacterial Growth Methods: Bacterial growth culture methods, and associated materials and conditions, are disclosed for respective species as follows:

Acinetobacter calcoaceticus (DSMZ # 1139) was obtained from the German Collection of Microorganisms and Cell Cultures (Braunschweig, Germany) as a vacuum dried culture. Cultures were then resuspended in Brain Heart Infusion (BHI) Broth (RPI Corp, Mt. Prospect, Ill., USA). Serial dilutions of the resuspended A. calcoaceticus culture were made into BHI and were allowed to grow for aerobically for 48 hours at 37° C. at 250 rpm until saturated.

Bacillus subtilis was a gift from the Gill lab (University of Colorado at Boulder) and was obtained as an actively growing culture. Serial dilutions of the actively growing B. subtilis culture were made into Luria Broth (RPI Corp, Mt. Prospect, Ill., USA) and were allowed to grow for aerobically for 24 hours at 37° C. at 250 rpm until saturated.

Chlorobium limicola (DSMZ# 245) was obtained from the German Collection of Microorganisms and Cell Cultures (Braunschweig, Germany) as a vacuum dried culture. Cultures were then resuspended using Pfennig's Medium I and II (#28 and 29) as described per DSMZ instructions. C. limicola was grown at 25° C. under constant vortexing.

Citrobacter braakii (DSMZ # 30040) was obtained from the German Collection of Microorganisms and Cell Cultures (Braunschweig, Germany) as a vacuum dried culture. Cultures were then resuspended in Brain Heart Infusion(BHI) Broth (RPI Corp, Mt. Prospect, Ill., USA). Serial dilutions of the resuspended C. braakii culture were made into BHI and were allowed to grow for aerobically for 48 hours at 30° C. at 250 rpm until saturated.

Clostridium acetobutylicum (DSMZ # 792) was obtained from the German Collection of Microorganisms and Cell Cultures (Braunschweig, Germany) as a vacuum dried culture. Cultures were then resuspended in Clostridium acetobutylicum medium (#411) as described per DSMZ instructions. C. acteobutylicum was grown anaerobically at 37° C. at 250 rpm until saturated.

Clostridium aminobutyricum (DSMZ # 2634) was obtained from the German Collection of Microorganisms and Cell Cultures (Braunschweig, Germany) as a vacuum dried culture. Cultures were then resuspended in Clostridium aminobutyricum medium (#286) as described per DSMZ instructions. C. aminobutyricum was grown anaerobically at 37° C. at 250 rpm until saturated.

Clostridium kluyveri (DSMZ #555) was obtained from the German Collection of Microorganisms and Cell Cultures (Braunschweig, Germany) as an actively growing culture. Serial dilutions of C. kluyveri culture were made into Clostridium kluyveri medium (#286) as described per DSMZ instructions. C. kluyveri was grown anaerobically at 37° C. at 250 rpm until saturated.

Cupriavidus metallidurans (DMSZ # 2839) was obtained from the German Collection of Microorganisms and Cell Cultures (Braunschweig, Germany) as a vacuum dried culture. Cultures were then resuspended in Brain Heart Infusion (BHI) Broth (RPI Corp, Mt. Prospect, Ill., USA). Serial dilutions of the resuspended C. metallidurans culture were made into BHI and were allowed to grow for aerobically for 48 hours at 30° C. at 250 rpm until saturated.

Cupriavidus necator (DSMZ # 428) was obtained from the German Collection of Microorganisms and Cell Cultures (Braunschweig, Germany) as a vacuum dried culture. Cultures were then resuspended in Brain Heart Infusion (BHI) Broth (RPI Corp, Mt. Prospect, Ill., USA). Serial dilutions of the resuspended C. necator culture were made into BHI and were allowed to grow for aerobically for 48 hours at 30° C. at 250 rpm until saturated.

Desulfovibrio fructosovorans (DSMZ # 3604) was obtained from the German Collection of Microorganisms and Cell Cultures (Braunschweig, Germany) as a vacuum dried culture. Cultures were then resuspended in Desulfovibrio fructosovorans medium (#63) as described per DSMZ instructions. D. fructosovorans was grown anaerobically at 37° C. at 250 rpm until saturated.

Escherichia coli Crooks (DSMZ#1576) was obtained from the German Collection of Microorganisms and Cell Cultures (Braunschweig, Germany) as a vacuum dried culture. Cultures were then resuspended in Brain Heart Infusion (BHI) Broth (RPI Corp, Mt. Prospect, Ill., USA). Serial dilutions of the resuspended E. coli Crooks culture were made into BHI and were allowed to grow for aerobically for 48 hours at 37° C. at 250 rpm until saturated.

Escherichia coli K12 was a gift from the Gill lab (University of Colorado at Boulder) and was obtained as an actively growing culture. Serial dilutions of the actively growing E. coli K12 culture were made into Luria Broth (RPI Corp, Mt. Prospect, Ill., USA) and were allowed to grow for aerobically for 24 hours at 37° C. at 250 rpm until saturated.

Halobacterium salinarum (DSMZ# 1576) was obtained from the German Collection of Microorganisms and Cell Cultures (Braunschweig, Germany) as a vacuum dried culture. Cultures were then resuspended in Halobacterium medium (#97) as described per DSMZ instructions. H. salinarum was grown aerobically at 37° C. at 250 rpm until saturated.

Lactobacillus delbrueckii (#4335) was obtained from WYEAST USA (Odell, Oreg., USA) as an actively growing culture. Serial dilutions of the actively growing L. delbrueckii culture were made into Brain Heart Infusion (BHI) broth (RPI Corp, Mt. Prospect, Ill., USA) and were allowed to grow for aerobically for 24 hours at 30° C. at 250 rpm until saturated.

Metallosphaera sedula (DSMZ #5348) was obtained from the German Collection of Microorganisms and Cell Cultures (Braunschweig, Germany) as an actively growing culture. Serial dilutions of M. sedula culture were made into Metallosphaera medium (#485) as described per DSMZ instructions. M. sedula was grown aerobically at 65° C. at 250 rpm until saturated.

Propionibacterium freudenreichii subsp. shermanii (DSMZ# 4902) was obtained from the German Collection of Microorganisms and Cell Cultures (Braunschweig, Germany) as a vacuum dried culture. Cultures were then resuspended in PYG-medium (#104) as described per DSMZ instructions. P. freudenreichii subsp. shermanii was grown anaerobically at 30° C. at 250 rpm until saturated.

Pseudomonas putida was a gift from the Gill lab (University of Colorado at Boulder) and was obtained as an actively growing culture. Serial dilutions of the actively growing P. putida culture were made into Luria Broth (RPI Corp, Mt. Prospect, Ill., USA) and were allowed to grow for aerobically for 24 hours at 37° C. at 250 rpm until saturated.

Streptococcus mutans (DSMZ# 6178) was obtained from the German Collection of Microorganisms and Cell Cultures (Braunschweig, Germany) as a vacuum dried culture. Cultures were then resuspended in Luria Broth (RPI Corp, Mt. Prospect, Ill., USA). S. mutans was grown aerobically at 37° C. at 250 rpm until saturated.

### Subsection II: Gel Preparation, DNA Separation, Extraction, Ligation, and Transformation Methods:

Molecular biology grade agarose (RPI Corp, Mt. Prospect, Ill., USA) was added to 1×TAE to make a 1% Agarose: TAE solution. To obtain 50×TAE add the following to 900 mL of distilled water: add the following to 900 ml distilled H₂O: 242 g Tris base (RPI Corp, Mt. Prospect, Ill., USA), 57.1 ml Glacial Acetic Acid (Sigma-Aldrich, St. Louis, Mo., USA) and 18.6 g EDTA (Fisher Scientific, Pittsburgh, Pa. USA) and adjust volume to 1 L with additional distilled water. To obtain 1×TAE, add 20 mL of 50×TAE to 980 mL of distilled water. The agarose-TAE solution was then heated until boiling occurred and the agarose was fully dissolved. The solution was allowed to cool to 50° C. before 10 mg/mL ethidium bromide (Acros Organics, Morris Plains, N.J., USA) was added at a concentration of 5 ul per 100 mL of 1% agarose solution. Once the ethidium bromide was added, the solution was briefly mixed and poured into a gel casting tray with the appropriate number of combs (Idea Scientific Co., Minneapolis, Minn., USA) per sample analysis. DNA samples were then mixed accordingly with 5×TAE loading buffer. 5×TAE loading buffer consists of 5×TAE (diluted from 50×TAE as described above), 20% glycerol (Acros Organics, Morris Plains, N.J., USA), 0.125% Bromophenol Blue (Alfa Aesar, Ward Hill, Mass., USA), and adjust volume to 50 mL with distilled water. Loaded gels were then run in gel rigs (Idea Scientific Co., Minneapolis, Minn., USA) filled with 1×TAE at a constant voltage of 125 volts for 25-30 minutes. At this point, the gels were removed from the gel boxes with voltage and visualized under a UV transilluminator (FOTODYNE Inc., Hartland, Wis., USA).

The DNA isolated through gel extraction was then extracted using the QIAquick Gel Extraction Kit following manufacturer's instructions (Qiagen (Valencia Calif. USA)). Similar methods are known to those skilled in the art.

The thus-extracted DNA then may be ligated into pSMART (Lucigen Corp, Middleton, Wis., USA), StrataClone (Stratagene, La Jolla, Calif., USA) or pCR2.1—TOPO TA (Invitrogen Corp, Carlsbad, Calif., USA) according to manufacturer's instructions. These methods are described in the next subsection of Common Methods.

Ligation Methods:

For ligations into pSMART vectors:

Gel extracted DNA was blunted using PCRTerminator (Lucigen Corp, Middleton, Wis., USA) according to manufacturer's instructions. Then 500 ng of DNA was added to 2.5 ul 4× CloneSmart vector premix, 1 ul CloneSmart DNA ligase (Lucigen Corp, Middleton, Wis., USA) and distilled water was added for a total volume of 10 ul. The reaction was then allowed to sit at room temperature for 30 minutes and then heat inactivated at 70° C. for 15 minutes and then placed on ice. E. cloni 10G Chemically Competent cells (Lucigen Corp, Middleton, Wis., USA) were thawed for 20 minutes on ice. 40 ul of chemically competent cells were placed into a microcentrifuge tube and 1 ul of heat inactivated CloneSmart Ligation was added to the tube. The whole reaction was stirred briefly with a pipette tip. The ligation and cells were incubated on ice for 30 minutes and then the cells were heat shocked for 45 seconds at 42° C. and then put back onto ice for 2 minutes. 960 ul of room temperature Recovery media (Lucigen Corp, Middleton, Wis., USA) and places into microcentrifuge tubes. Shake tubes at 250 rpm for 1 hour at 37° C. Plate 100 ul of transformed cells on Luria Broth plates (RPI Corp, Mt. Prospect, Ill., USA) plus appropriate antibiotics depending on the pSMART vector used. Incubate plates overnight at 37° C.

For ligations into StrataClone:

Gel extracted DNA was blunted using PCRTerminator (Lucigen Corp, Middleton, Wis., USA) according to manufacturer's instructions. Then 2 ul of DNA was added to 3 ul StrataClone Blunt Cloning buffer and 1 ul StrataClone Blunt vector mix amp/kan (Stratagene, La Jolla, Calif., USA) for a total of 6 ul. Mix the reaction by gently pipeting up at down and incubate the reaction at room temperature for 30 minutes then place onto ice. Thaw a tube of StrataClone chemically competent cells (Stratagene, La Jolla, Calif., USA) on ice for 20 minutes. Add 1 ul of the cloning reaction to the tube of chemically competent cells and gently mix with a pipette tip and incubate on ice for 20 minutes. Heat shock the transformation at 42° C. for 45 seconds then put on ice for 2 minutes. Add 250 ul pre-warmed Luria Broth (RPI Corp, Mt. Prospect, Ill., USA) and shake at 250 rpm for 37° C. for 2 hour. Plate 100 ul of the transformation mixture onto Luria Broth plates (RPI Corp, Mt. Prospect, Ill., USA) plus appropriate antibiotics. Incubate plates overnight at 37° C.

For Ligations into pCR2.1—TOPO TA:

Add 1 ul TOPO vector, 1 ul Salt Solution (Invitrogen Corp, Carlsbad, Calif., USA) and 3 ul gel extracted DNA into a microcentrifuge tube. Allow the tube to incubate at room temperature for 30 minutes then place the reaction on ice. Thaw one tube of TOP10F′ chemically competent cells (Invitrogen Corp, Carlsbad, Calif., USA) per reaction. Add 1 ul of reaction mixture into the thawed TOP10F′ cells and mix gently by swirling the cells with a pipette tip and incubate on ice for 20 minutes. Heat shock the transformation at 42° C. for 45 seconds then put on ice for 2 minutes. Add 250 ul pre-warmed SOC media (Invitrogen Corp, Carlsbad, Calif., USA) and shake at 250 rpm for 37° C. for 1 hour. Plate 100 ul of the transformation mixture onto Luria Broth plates (RPI Corp, Mt. Prospect, Ill., USA) plus appropriate antibiotics. Incubate plates overnight at 37° C.

General Transformation and Related Culture Methodologies:

Chemically competent transformation protocols are carried out according to the manufactures instructions or according to the literature contained in Molecular Cloning (Sambrook and Russell). Generally, plasmid DNA or ligation products are chilled on ice for 5 to 30 min. in solution with chemically competent cells. Chemically competent cells are a widely used product in the field of biotechnology and are available from multiple vendors, such as those indicated above in this Subsection. Following the chilling period cells generally are heat-shocked for 30 seconds at 42° C. without shaking, re-chilled and combined with 250 microliters of rich media, such as S.O.C. Cells are then incubated at 37° C. while shaking at 250 rpm for 1 hour. Finally, the cells are screened for successful transformations by plating on media containing the appropriate antibiotics.

The choice of an E. coli host strain for plasmid transformation is determined by considering factors such as plasmid stability, plasmid compatibility, plasmid screening methods and protein expression. Strain backgrounds can be changed by simply purifying plasmid DNA as described above and transforming the plasmid into a desired or otherwise appropriate E. coli host strain such as determined by experimental necessities, such as any commonly used cloning strain (e.g., DH5a, Top10F′, E. cloni 10G, etc.).

### Subsection III. HPLC Analytical Method

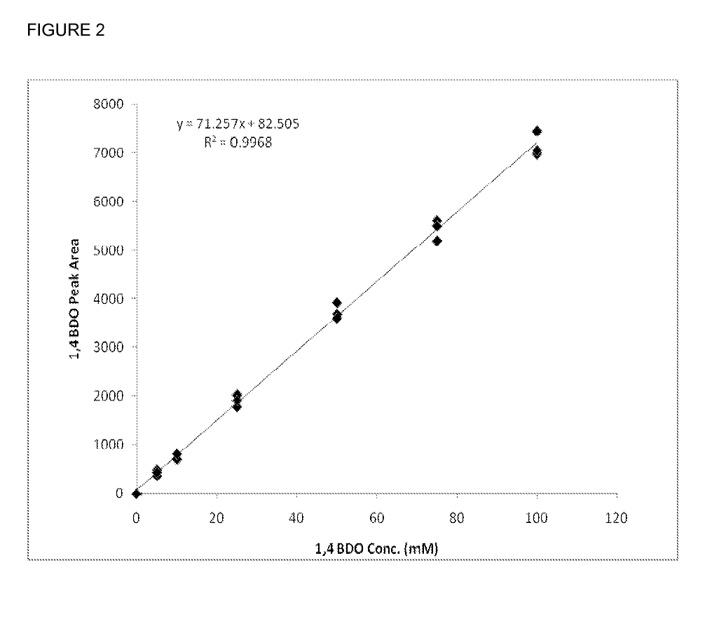

The Waters chromatography system (Milford, Mass.) consisted of the following: 600S Controller, 616 Pump, 717 Plus Autosampler, 410 Refractive Index (RI) Detector, and an in-line mobile phase Degasser. In addition, an Eppendorf external column heater was used and the data were collected using an SRI (Torrance, Calif.) analog-to-digital converter linked to a standard desk top computer. Data were analyzed using the SRI Peak Simple software. A Coregel Ion310 ion exclusion column (Transgenomic, Inc., San Jose, Calif.) was employed. The column resin was a sulfonated polystyrene divinyl benzene with a particle size of 8 μm and column dimensions were 150×6.5 mm. The mobile phase consisted of sulfuric acid (Fisher Scientific, Pittsburgh, Pa. USA) diluted with deionized (18 MΩcm) water to a concentration of 0.02 N and vacuum filtered through a 0.2 μm nylon filter. The flow rate of the mobile phase was 0.6 mL/min. The RI detector was operated at a sensitivity of 128 and the column was heated to 60° C. The same equipment and method as described herein is used for 1,4-BDO analyses for relevant general examples. Calibration curves using this HPLC method with a 1,4-BDO reagent grade standard (Sigma-Aldrich, St. Louis, Mo., USA) is provided in FIG. 2.

### Summary of Suppliers Section

This section is provided for a summary of suppliers, and may be amended to incorporate additional supplier information in subsequent filings. The names and city addresses of major suppliers are provided in the methods above. In addition, as to Qiagen products, the DNeasy® Blood and Tissue Kit, Cat. No. 69506, is used in the methods for genomic DNA preparation; the QIAprep® Spin (“mini prep”), Cat. No. 27106, is used for plasmid DNA purification, and the QIAquick® Gel Extraction Kit, Cat. No. 28706, is used for gel extractions as described above.

### Bio-Production Media

Bio-production media, which is used in the present invention with recombinant microorganisms having a biosynthetic pathway for 1,4-BDO, must contain suitable carbon substrates. Suitable substrates may include, but are not limited to, monosaccharides such as glucose and fructose, oligosaccharides such as lactose or sucrose, polysaccharides such as starch or cellulose or mixtures thereof and unpurified mixtures from renewable feedstocks such as cheese whey permeate, cornsteep liquor, sugar beet molasses, and barley malt. Additionally the carbon substrate may also be one-carbon substrates such as carbon dioxide, or methanol for which metabolic conversion into key biochemical intermediates has been demonstrated. In addition to one and two carbon substrates methylotrophic organisms are also known to utilize a number of other carbon containing compounds such as methylamine, glucosamine and a variety of amino acids for metabolic activity. For example, methylotrophic yeast are known to utilize the carbon from methylamine to form trehalose or glycerol (Bellion et al., Microb. Growth C1-Compd., [Int. Symp.], 7th (1993), 415-32. Editor(s): Murrell, J. Collin; Kelly, Don P. Publisher: Intercept, Andover, UK). Similarly, various species of Candida will metabolize alanine or oleic acid (Sulter et al., Arch. Microbiol. 153:485-489 (1990)). Hence it is contemplated that the source of carbon utilized in the present invention may encompass a wide variety of carbon containing substrates and will only be limited by the choice of organism.

Although it is contemplated that all of the above mentioned carbon substrates and mixtures thereof are suitable in the present invention, common carbon substrates are glucose, fructose, and sucrose, as well as mixtures of any of these sugars. Sucrose may be obtained from feedstocks such as sugar cane, sugar beets, cassaya, and sweet sorghum. Glucose and dextrose may be obtained through saccharification of starch based feedstocks including grains such as corn, wheat, rye, barley, and oats.

In addition, fermentable sugars may be obtained from cellulosic and lignocellulosic biomass through processes of pretreatment and saccharification, as described, for example, in co-owned and co-pending US patent application US20070031918A1, which is herein incorporated by reference. Biomass refers to any cellulosic or lignocellulosic material and includes materials comprising cellulose, and optionally further comprising hemicellulose, lignin, starch, oligosaccharides and/or monosaccharides. Biomass may also comprise additional components, such as protein and/or lipid. Biomass may be derived from a single source, or biomass can comprise a mixture derived from more than one source; for example, biomass could comprise a mixture of corn cobs and corn stover, or a mixture of grass and leaves. Biomass includes, but is not limited to, bioenergy crops, agricultural residues, municipal solid waste, industrial solid waste, sludge from paper manufacture, yard waste, wood and forestry waste. Examples of biomass include, but are not limited to, corn grain, corn cobs, crop residues such as corn husks, corn stover, grasses, wheat, wheat straw, barley, barley straw, hay, rice straw, switchgrass, waste paper, sugar cane bagasse, sorghum, soy, components obtained from milling of grains, trees, branches, roots, leaves, wood chips, sawdust, shrubs and bushes, vegetables, fruits, flowers and animal manure.

In addition to an appropriate carbon source, bio-production media must contain suitable minerals, salts, cofactors, buffers and other components, known to those skilled in the art, suitable for the growth of the cultures and promotion of the enzymatic pathway necessary for 1,4-BDO production.

Culture Conditions

Typically cells are grown at a temperature in the range of about 25° C. to about 40° C. in an appropriate medium. Suitable growth media in the present invention are common commercially prepared media such as Luria Bertani (LB) broth, M9 minimal media, Sabouraud Dextrose (SD) broth, Yeast medium (YM) broth or (Ymin) yeast synthetic minimal media. Other defined or synthetic growth media may also be used, and the appropriate medium for growth of the particular microorganism will be known by one skilled in the art of microbiology or bio-production science.

Suitable pH ranges for the bio-production are between pH 5.0 to pH 9.0, where pH 6.0 to pH 8.0 is a typical pH range for the initial condition.

Bio-productions may be performed under aerobic, microaerobic, or anaerobic conditions.

The amount of 1,4-BDO produced in the bio-production medium generally can be determined using a number of methods known in the art, for example, high performance liquid chromatography (HPLC) or gas chromatography (GC). Specific HPLC methods for the specific examples are provided herein.

The above discloses and teaches methods, compositions, and systems that provide for production of 1,4-BDO. It is appreciated that as the titer of 1,4-BDO gets higher it exerts a growth-inhibiting and/or toxic effect on microorganisms in the respective culture or industrial system. Any of a number of approaches may be employed to determine the cause(s) and mechanism(s) of such undesired effect(s), and/or to identify genes and/or nucleic acid sequences, that when expressed, result in greater tolerance to 1,4-BDO. For example, directed selection, non-directed selection, and/or identification of naturally tolerance colonies or strains may be utilized, such as is summarized above. Also, among the genomics approaches to identifying such tolerance-related genes and/or nucleic acid sequences is a method described in U.S. Provisional Application No. 60/611,377 filed Sep. 20, 2004 and U.S. patent application Ser. No. 11/231,018 filed Sep. 20, 2005, both entitled: “Mixed-Library Parallel Gene Mapping Quantitation Microarray Technique for Genome Wide Identification of Trait Conferring Genes” (hereinafter, the “Gill et al. Technique”), which are incorporated herein by reference in their entirety for the teaching of the technique.

Accordingly the present inventors conceive that the referenced Gill et al. technique, and/or other techniques, may be utilized to supply data that may then be analyzed to identify genetic elements, and/or to learn of non-genetic modifications that may be made in a culture or industrial system, to increase the tolerance of a microorganism to 1,4-BDO as well as the productivity and yield of 1,4-BDO by a microorganism in a bio-production system. The present inventors further conceive that the tolerance-improving productivity as well as yield enhancing approaches thereby identified and developed may be incorporated into a recombinant microorganism comprising any of the 1,4-BDO production pathways described and/or taught herein, to provide a recombinant microorganism that both produces and has increased tolerance to as well as productivity of yield of (compared with a non-modified control microorganism) 1,4-BDO. Such ‘doubly-modified’ recombinant microorganism may be appreciated to have high commercial value for use in industrial systems that are designed to biosynthesize 1,4-BDO in a cost-effective manner. It is well appreciated that higher tolerances and final titers to an end product of interest results in relatively lower downstream separation and liquids-transfer costs.

### Example 26

### Determination of MIC for 1,4-BDO in Control Microorganism

Overnight cultures of E. coli K12 were started in 5 mL LB containing no antibiotic 12-16 hours before the experiment was performed. LB broth was inoculated using aseptic technique. The culture was incubated overnight in a shaking incubator at 37 C.

The next morning 10 mL of M9 was inoculated with 100 μL of cells from the overnight culture. The tube was inverted to mix the cells prior to incubation at 37 C in the shaking incubator. While the cells were growing 96 well plates were prepared for inoculation.

A 237 g/L stock solution of 1,4-Butanediol was made by combining 4.92 mL of 1,4 BDO with 10 mL water. The solutions pH was checked using pH paper; it was acidic. The solution was made to be at a neutral pH of 7.0 by adding 1M NaOH. This was achieved by adding approximately 22 μL of 1M NaOH to the stock solution. The solution was then vortexed to mix. The pH of the solution was then checked again using pH paper. More 1M NaOH was added to the solution in 2 μL increments, vortexing the solution after addition of more 1M NaOH and then checking the pH of the solution again with pH paper. This was continued until the solution had a pH of 7.0.

A solution of concentrated M9 was made by combining 4 mL of 5×M9 salts, 400 μL of 20% glucose, 40 μL 1M MgSO4, and 2 μL of 1M CaCl₂.

The plate was loaded as follows: 45 μL of concentrated M9 mixture was added to each well containing the compound concentrations and the following dilutions were performed:

Dilution Scheme for 1,4-BDO

| | | Concentration | | Final Conc |
|---|---|---|---|---|
| N/A | Amt of 1,4-BDO | Of Stock Soln | Amt of H₂0 | in the Well |
| 1 | 68 μL | 237 g/L (Stock) | 67 μL | 80 g/L |
| 2 | 59 μL | 237 g/L (Stock) | 76 μL | 70 g/L |
| 3 | 51 μL | 237 g/L (Stock) | 84 μL | 60 g/L |
| 4 | 42 μL | 237 g/L (Stock) | 93 μL | 50 g/L |
| 5 | 34 μL | 237 g/L (Stock) | 101 μL | 40 g/L |
| 6 | 25 μL | 237 g/L (Stock) | 110 μL | 30 g/L |
| 7 | 17 μL | 237 g/L (Stock) | 118 μL | 20 g/L |
| 8 | 9 μL | 237 g/L (Stock) | 126 μL | 11 g/L |

Controls were prepared and loaded onto the plate as follows: 135 μL of H₂O was added to positive control wells. 45 μL of the concentrated M9 mixture was added to each positive control well. 200 μL of water was added to each negative control well.

The OD₆₀₀ of the cells from the overnight culture that was inoculated into M9 was checked using the spectrophotometer. The final OD₆₀₀ of the cells was between 0.195 and 0.200. To achieve a final OD within this range the spectrophotomter was blanked with water. 1 mL of the overnight/M9 culture was added to the cuvette which was then placed into the spectrophotometer. The cells were then diluted down to the proper concentration by adding approximately 100 μL of M9 and pipetting the solution up and down to mix. Since the OD₆₀₀ was not between 0.195 and 2.00 the cells were diluted further in the same manner until the final concentration of the cells was reached. After the cells were at the proper OD a 1:50 dilution was performed into M9. 20 μL of cells from the 1:50 dilution was added to each positive control well and also to each well for each concentration containing the chemical that is being tested. The plate was covered with an aluminum foil plate sealer and placed in the 37 C incubator. The MIC was checked at 24 hours by visually inspecting the plate. The MIC endpoint was the lowest concentration of compound at which there was no visible growth.

The minimum inhibitory concentration (MIC) for 1,4-Butanediol was determined per the method. Three separate samples were made and tested on separate days. The MIC value for each of the three replicates was 50 g/L for 1,4-BDO tested with E. coli K12 control microorganisms.

This MIC procedure may be used for comparisons of microorganisms having differing levels of tolerance to 1,4-BDO, toward identifying more 1,4-BDO-tolerant microorganisms and their genetic elements.

### Bio-Production Reactors and Systems:

Any of the recombinant microorganisms as described and/or referred to above may be introduced into an industrial bio-production system where the microorganisms convert a carbon source into 1,4-BDO in a commercially viable operation. The bio-production system includes the introduction of such a recombinant microorganism into a bioreactor vessel, with a carbon source substrate and bio-production media suitable for growing the recombinant microorganism, and maintaining the bio-production system within a suitable temperature range (and dissolved oxygen concentration range if the reaction is aerobic or microaerobic) for a suitable time to obtain a desired conversion of a portion of the substrate molecules to 1,4-BDO. In some instances, the quantity of 1,4-BDO produced in the bioreactor vessel is a measurable quantity. Industrial bio-production systems and their operation are well-known to those skilled in the arts of chemical engineering and bioprocess engineering.

In some instances, the bio-production system is microbial bioreactor. In some instances, the microbial bioreactor comprises a bioreactor vessel. In some instances, the microbial bioreactor comprises a carbon source. In some instances, the microbial bioreactor comprises one or more recombinant microorganism described herein. In some instances, the microbial bioreactor comprises media. In some instances, the microbial bioreactor is an analytical-scale microbial bioreactor. In some instances, the microbial bioreactor is a small-scale microbial bioreactor. In some instances, the microbial bioreactor is a medium-scale microbial bioreactor. In some instances, the microbial bioreactor is a large-scale microbial bioreactor. In some instances, the microbial bioreactor is an industrial-scale microbial bioreactor.

In some instances, the media is minimal media.

The following paragraphs provide an overview of the methods and aspects of industrial systems that may be used for the bio-production of 1,4-BDO.

In various embodiments, any of a wide range of sugars, including, but not limited to sucrose, glucose, xylose, cellulose or hemixellulose, are provided to a microorganism as a carbon source, such as in an industrial system comprising a reactor vessel in which a defined media (such as a minimal salts media including but not limited to M9 minimal media, potassium sulfate minimal media, yeast synthetic minimal media and many others or variations of these), an inoculum of a microorganism providing one or more of the 1,4-BDO biosynthetic pathway alternatives, and the a carbon source may be combined. The carbon source enters the cell and is catabolized by well-known and common metabolic pathways to yield common metabolic intermediates, including phosphoenolpyruvate (PEP). (See Molecular Biology of the Cell, 3^{rd} Ed., B. Alberts et al. Garland Publishing, New York, 1994, pp. 42-45, 66-74, incorporated by reference for the teachings of basic metabolic catabolic pathways for sugars; Principles of Biochemistry, 3^{rd} Ed., D. L. Nelson & M. M. Cox, Worth Publishers, New York, 2000, pp 527-658, incorporated by reference for the teachings of major metabolic pathways; and Biochemistry, 4^{th} Ed., L. Stryer, W. H. Freeman and Co., New York, 1995, pp. 463-650, also incorporated by reference for the teachings of major metabolic pathways.). The appropriate intermediates are subsequently converted to 1,4-BDO by one or more of the above-disclosed biosynthetic pathways. Further to types of industrial bio-production, various embodiments of the present invention may employ a batch type of industrial bioreactor. A classical batch bioreactor system is considered “closed” meaning that the composition of the medium is established at the beginning of a respective bio-production event and not subject to artificial alterations and additions during the time period ending substantially with the end of the bio-production event. Thus, at the beginning of the bio-production event the medium is inoculated with the desired organism or organisms, and bio-production is permitted to occur without adding anything to the system. Typically, however, a “batch” type of bio-production event is batch with respect to the addition of carbon source and attempts are often made at controlling factors such as pH and oxygen concentration. In batch systems the metabolite and biomass compositions of the system change constantly up to the time the bio-production event is stopped. Within batch cultures cells moderate through a static lag phase to a high growth log phase and finally to a stationary phase where growth rate is diminished or halted. If untreated, cells in the stationary phase will eventually die. Cells in log phase generally are responsible for the bulk of production of a desired end product or intermediate.

A variation on the standard batch system is the Fed-Batch system. Fed-Batch bio-production processes are also suitable in the present invention and comprise a typical batch system with the exception that the substrate is added in increments as the bio-production progresses. Fed-Batch systems are useful when catabolite repression is apt to inhibit the metabolism of the cells and where it is desirable to have limited amounts of substrate in the media. Measurement of the actual substrate concentration in Fed-Batch systems may be measured directly, such as by sample analysis at different times, or estimated on the basis of the changes of measurable factors such as pH, dissolved oxygen and the partial pressure of waste gases such as CO₂. Batch and Fed-Batch approaches are common and well known in the art and examples may be found in Thomas D. Brock in Biotechnology: A Textbook of Industrial Microbiology, Second Edition (1989) Sinauer Associates, Inc., Sunderland, Mass., Deshpande, Mukund V., Appl. Biochem. Biotechnol., 36:227, (1992), and Biochemical Engineering Fundamentals, 2^{nd} Ed. J. E. Bailey and D. F. 011 is, McGraw Hill, New York, 1986, herein incorporated by reference for general instruction on bio-production, which as used herein may be aerobic, microaerobic, or anaerobic.

Although the present invention may be performed in fed-batch mode it is contemplated that the method would be adaptable to continuous bio-production methods. Continuous bio-production is considered an “open” system where a defined bio-production medium is added continuously to a bioreactor and an equal amount of conditioned media is removed simultaneously for processing. Continuous bio-production generally maintains the cultures within a controlled density range where cells are primarily in log phase growth. Two types of continuous bioreactor operation include: 1) Chemostat—where fresh media is fed to the vessel while simultaneously removing an equal rate of the vessel contents. The limitation of this approach is that cells are lost and high cell density generally is not achievable. In fact, typically one can obtain much higher cell density with a fed-batch process. 2) Perfusion culture, which is similar to the chemostat approach except that the stream that is removed from the vessel is subjected to a separation technique which recycles viable cells back to the vessel. This type of continuous bioreactor operation has been shown to yield significantly higher cell densities than fed-batch and can be operated continuously. Continuous bio-production is particularly advantageous for industrial operations because it has less down time associated with draining, cleaning and preparing the equipment for the next bio-production event. Furthermore, it is typically more economical to continuously operate downstream unit operations, such as distillation, than to run them in batch mode.

Continuous bio-production allows for the modulation of one factor or any number of factors that affect cell growth or end product concentration. For example, one method will maintain a limiting nutrient such as the carbon source or nitrogen level at a fixed rate and allow all other parameters to moderate. In other systems a number of factors affecting growth can be altered continuously while the cell concentration, measured by media turbidity, is kept constant. Continuous systems strive to maintain steady state growth conditions and thus the cell loss due to the medium being drawn off must be balanced against the cell growth rate in the bio-production. Methods of modulating nutrients and growth factors for continuous bio-production processes as well as techniques for maximizing the rate of product formation are well known in the art of industrial microbiology and a variety of methods are detailed by Brock, supra.

It is contemplated that embodiments of the present invention may be practiced using either batch, fed-batch or continuous processes and that any known mode of bio-production would be suitable. Additionally, it is contemplated that cells may be immobilized on an inert scaffold as whole cell catalysts and subjected to suitable bio-production conditions for 1,4-BDO production.

The following published resources are incorporated by reference herein for their respective teachings to indicate the level of skill in these relevant arts, and as needed to support a disclosure that teaches how to make and use methods of industrial bio-production of 1,4-BDO from sugar sources, and also industrial systems that may be used to achieve such conversion with any of the recombinant microorganisms of the present invention (Biochemical Engineering Fundamentals, 2^{nd} Ed. J. E. Bailey and D. F. 011 is, McGraw Hill, New York, 1986, entire book for purposes indicated and Chapter 9, pages 533-657 in particular for biological reactor design; Unit Operations of Chemical Engineering, 5^{th} Ed., W. L. McCabe et al., McGraw Hill, New York 1993, entire book for purposes indicated, and particularly for process and separation technologies analyses; Equilibrium Staged Separations, P. C. Wankat, Prentice Hall, Englewood Cliffs, N.J. USA, 1988, entire book for separation technologies teachings).

### Conversions of 1,4-BDO to Other Products

1,4-BDO is recognized in the art of polymer chemistry as a versatile intermediate. This is due to its terminal, primary hydroxyl groups and its general hydrophilic nature. 1,4-BDO may be utilized in many polyurethane and polyester compositions such as when polymerization proceeds by reactions with diacids or diisocyanates.

Accordingly, polyesters comprising 1,4-BDO may be prepared by esterification reaction or ester exchange reaction between a dicarboxylic acid or an ester derivative thereof and a diol and subsequent polycondensation reaction. This is usually under a reduced pressure of 10 kPa or less while removing formed water and low-molecular weight materials such as diols out the system.

Further among its many uses, 1,4-BDO may be converted by known synthetic processes into γ-butyrolactone (GBL). Also, in the presence of phosphoric acid and high temperature, 1,4-BDO dehydrates to the important solvent tetrahydrofuran (Ethers, by Lawrence Karas and W. J. Piel, in Kirk-Othmer Encyclopedia of Chemical Technology. (2004). John Wiley & Sons, Inc., incorporated by reference for the method of production of tetrahydrofuran using 1,4-BDO). Alternatively, at about 200° C. in the presence of soluble ruthenium catalysts, 1,4-BDO undergoes dehydrogenation to form butyrolactone (J. Zhao, J. F. Hartwig “Acceptorless, Neat, Ruthenium-Catalyzed Dehydrogenative Cyclization of Diols to Lactones” Organometallics 2005, volume 24, 2441-2446, incorporated by reference for its teachings of the noted method of conversion of 1,4-BDO to butyrolactone.

Thus, in accordance with aspects of the present invention, 1,4-BBO is produced by any of the bio-production pathways in any of the microorganisms referenced herein, and the 1,4-BDO so produced, and thereafter separated by means known to those skilled in the art, is further reacted to form any of the downstream products described in this section, and/or more generally known to those skilled in the art.

The scope of the present invention is not meant to be limited to the exact sequences provided herein. It is appreciated that a range of modifications to nucleic acid and to amino acid sequences may be made and still provide a desired functionality. The following discussion is provided to more clearly define ranges of variation that may be practiced and still remain within the scope of the present invention.

It is recognized in the art that some amino acid sequences of the present invention can be varied without significant effect of the structure or function of the proteins disclosed herein. Variants included can constitute deletions, insertions, inversions, repeats, and type substitutions so long as the indicated enzyme activity is not significantly affected. Guidance concerning which amino acid changes are likely to be phenotypically silent can be found in Bowie, J. U., et Al., “Deciphering the Message in Protein Sequences: Tolerance to Amino Acid Substitutions,” Science 247:1306-1310 (1990).

In various embodiments polypeptides obtained by the expression of the polynucleotide molecules of the present invention may have at least approximately 80%, 90%, 95%, 96%, 97%, 98%, 99% or 100% identity to one or more amino acid sequences encoded by the genes and/or nucleic acid sequences described herein for the 1,4-BDO biosynthesis pathways. A truncated respective polypeptide has at least about 90% of the full length of a polypeptide encoded by a nucleic acid sequence encoding the respective native enzyme, and more particularly at least 95% of the full length of a polypeptide encoded by a nucleic acid sequence encoding the respective native enzyme. By a polypeptide having an amino acid sequence at least, for example, 95% “identical” to a reference amino acid sequence of a polypeptide is intended that the amino acid sequence of the claimed polypeptide is identical to the reference sequence except that the claimed polypeptide sequence can include up to five amino acid alterations per each 100 amino acids of the reference amino acid of the polypeptide. In other words, to obtain a polypeptide having an amino acid sequence at least 95% identical to a reference amino acid sequence, up to 5% of the amino acid residues in the reference sequence can be deleted or substituted with another amino acid, or a number of amino acids up to 5% of the total amino acid residues in the reference sequence can be inserted into the reference sequence. These alterations of the reference sequence can occur at the amino or carboxy terminal positions of the reference amino acid sequence or anywhere between those terminal positions, interspersed either individually among residues in the reference sequence or in one or more contiguous groups within the reference sequence.

As a practical matter, whether any particular polypeptide is at least 80%, 85%, 90%, 92%, 95%, 96%, 97%, 98% or 99% identical to any reference amino acid sequence of any polypeptide described herein (which may correspond with a particular nucleic acid sequence described herein), such particular polypeptide sequence can be determined conventionally using known computer programs such the Bestfit program (Wisconsin Sequence Analysis Package, Version 8 for Unix, Genetics Computer Group, University Research Park, 575 Science Drive, Madison, Wis. 53711). When using Bestfit or any other sequence alignment program to determine whether a particular sequence is, for instance, 95% identical to a reference sequence according to the present invention, the parameters are set, of course, such that the percentage of identity is calculated over the full length of the reference amino acid sequence and that gaps in homology of up to 5% of the total number of amino acid residues in the reference sequence are allowed.

For example, in a specific embodiment the identity between a reference sequence (query sequence, a sequence of the present invention) and a subject sequence, also referred to as a global sequence alignment, may be determined using the FASTDB computer program based on the algorithm of Brutlag et al. (Comp. App. Biosci. 6:237-245 (1990)). Preferred parameters used in a FASTDB amino acid alignment are: Scoring Scheme=PAM 0, k-tuple=2, Mismatch Penalty=1, Joining Penalty=20, Randomization Group Length=0, Cutoff Score=1, Window Size=sequence length, Gap Penalty=5, Gap Size Penalty=0.05, Window Size=500 or the length of the subject amino acid sequence, whichever is shorter. According to this embodiment, if the subject sequence is shorter than the query sequence due to N- or C-terminal deletions, not because of internal deletions, a manual correction is made to the results to take into consideration the fact that the FASTDB program does not account for N- and C-terminal truncations of the subject sequence when calculating global percent identity. For subject sequences truncated at the N- and C-termini, relative to the query sequence, the percent identity is corrected by calculating the number of residues of the query sequence that are N- and C-terminal of the subject sequence, which are not matched/aligned with a corresponding subject residue, as a percent of the total bases of the query sequence. A determination of whether a residue is matched/aligned is determined by results of the FASTDB sequence alignment. This percentage is then subtracted from the percent identity, calculated by the above FASTDB program using the specified parameters, to arrive at a final percent identity score. This final percent identity score is what is used for the purposes of this embodiment. Only residues to the N- and C-termini of the subject sequence, which are not matched/aligned with the query sequence, are considered for the purposes of manually adjusting the percent identity score. That is, only query residue positions outside the farthest N- and C-terminal residues of the subject sequence. For example, a 90 amino acid residue subject sequence is aligned with a 100 residue query sequence to determine percent identity. The deletion occurs at the N-terminus of the subject sequence and therefore, the FASTDB alignment does not show a matching/alignment of the first 10 residues at the N-terminus. The 10 unpaired residues represent 10% of the sequence (number of residues at the N- and C-termini not matched/total number of residues in the query sequence) so 10% is subtracted from the percent identity score calculated by the FASTDB program. If the remaining 90 residues were perfectly matched the final percent identity would be 90%. In another example, a 90 residue subject sequence is compared with a 100 residue query sequence. This time the deletions are internal deletions so there are no residues at the N- or C-termini of the subject sequence which are not matched/aligned with the query. In this case the percent identity calculated by FASTDB is not manually corrected. Once again, only residue positions outside the N- and C-terminal ends of the subject sequence, as displayed in the FASTDB alignment, which are not matched/aligned with the query sequence are manually corrected for.

Also as used herein, the term “homology” refers to the optimal alignment of sequences (either nucleotides or amino acids), which may be conducted by computerized implementations of algorithms. “Homology”, with regard to polynucleotides, for example, may be determined by analysis with BLASTN version 2.0 using the default parameters. “Homology”, with respect to polypeptides (i.e., amino acids), may be determined using a program, such as BLASTP version 2.2.2 with the default parameters, which aligns the polypeptides or fragments being compared and determines the extent of amino acid identity or similarity between them. It will be appreciated that amino acid “homology” includes conservative substitutions, i.e. those that substitute a given amino acid in a polypeptide by another amino acid of similar characteristics. Typically seen as conservative substitutions are the following replacements: replacements of an aliphatic amino acid such as Ala, Val, Leu and Ile with another aliphatic amino acid; replacement of a Ser with a Thr or vice versa; replacement of an acidic residue such as Asp or Glu with another acidic residue; replacement of a residue bearing an amide group, such as Asn or Gln, with another residue bearing an amide group; exchange of a basic residue such as Lys or Arg with another basic residue; and replacement of an aromatic residue such as Phe or Tyr with another aromatic residue. A polypeptide sequence (i.e., amino acid sequence) or a polynucleotide sequence comprising at least 50% homology to another amino acid sequence or another nucleotide sequence respectively has a homology of 50% or greater than 50%, e.g., 60%, 70%, 80%, 90% or 100%.

The above descriptions and methods for sequence homology are intended to be exemplary and it is recognized that this concept is well-understood in the art. Further, it is appreciated that nucleic acid sequences may be varied and still provide a functional enzyme, and such variations are within the scope of the present invention. Nucleic acid sequences that encode polypeptides that provide the indicated functions for 1,4-BDO production are considered within the scope of the present invention. These may be further defined by the stringency of hybridization, described below, but this is not meant to be limiting when a function of an encoded polypeptide matches a specified 1,4-BDO biosynthesis pathway enzyme activity.

Further to nucleic acid sequences, “hybridization” refers to the process in which two single-stranded polynucleotides bind non-covalently to form a stable double-stranded polynucleotide. The term “hybridization” may also refer to triple-stranded hybridization. The resulting (usually) double-stranded polynucleotide is a “hybrid” or “duplex.” “Hybridization conditions” will typically include salt concentrations of less than about 1M, more usually less than about 500 mM and less than about 200 mM. Hybridization temperatures can be as low as 5° C., but are typically greater than 22° C., more typically greater than about 30° C., and often are in excess of about 37° C. Hybridizations may be performed under stringent conditions, i.e. conditions under which a probe will hybridize to its specific target subsequence but, at a statistical level, not to relatively close sequences. Stringent conditions are sequence-dependent and are different in different circumstances. Longer fragments may require higher hybridization temperatures for specific hybridization. As other factors may affect the stringency of hybridization, including base composition and length of the complementary strands, presence of organic solvents and extent of base mismatching, the combination of parameters is more important than the absolute measure of any one alone. Generally, stringent conditions are selected to be about 5° C. lower than the Tₘ for the specific sequence at a defined ionic strength and pH. Exemplary stringent conditions include salt concentration of at least 0.01 M to no more than 1 M Na ion concentration (or other salts) at a pH 7.0 to 8.3 and a temperature of at least 25° C. For example, conditions of 5×SSPE (750 mM NaCl, 50 mM NaPhosphate, 5 mM EDTA, pH 7.4) and a temperature of 25-30° C. are suitable for allele-specific probe hybridizations.

Hybridizations also may be performed under selective conditions, i.e. conditions under which a probe will hybridize to its target subsequence and also, to some extent, to relatively close sequences. Selective conditions for hybridization are sequence-dependent and are different in different circumstances. Longer fragments may require higher hybridization temperatures for selective hybridization.

For various hybridization conditions, see for example, Sambrook and Russell and Anderson “Nucleic Acid Hybridization” 1^{st} Ed., BIOS Scientific Publishers Limited (1999), which are hereby incorporated by reference for hybridization protocols.

Having so described the present invention and provided examples, and further discussion, and in view of the above paragraphs, it is appreciated that various non-limiting aspects of the present invention may include a genetically modified (recombinant) microorganism comprising one or more nucleic acid sequences that encodes one or more polypeptides with at least 85%, 90%, 95%, 99% or 100% amino acid sequence identity to any of the enzymes of any of 1,4-BDO biosynthetic pathway B, wherein the one or more polypeptides have enzymatic activity effective to perform the enzymatic reaction of the respective 1,4-BDO biosynthetic pathway enzyme, and the recombinant microorganism biosynthesizes 1,4-BDO. For example, in one instance, the present invention contemplates a modified or recombinant microorganism comprising a nucleic acid encoding a polypeptide having acetyl-coA acetyltransferase activity, such as atoB or thiL. In another instance, the present invention contemplates a modified or recombinant microorganism comprising a nucleic acid encoding a polypeptide having β-hydroxybutyryl-CoA dehydrogenase activity, such as hbd. In another instance, the present invention contemplates a modified or recombinant microorganism comprising a nucleic acid encoding a polypeptide having crotonase activity, such as ech or crt. In another instance, the present invention contemplates a modified or recombinant microorganism comprising a nucleic acid encoding a polypeptide having vinylacetyl-CoA-A-isomerase and 4-hydroxybutyryl-CoA dehydratase activities, such as abfD. In another instance, the present invention contemplates a modified or recombinant microorganism comprising a nucleic acid encoding a polypeptide having 4-hydroxybutyrate-CoA-hydrolase activity, such as abfT. In another instance, the present invention contemplates a modified or recombinant microorganism comprising a nucleic acid encoding a polypeptide having 1,3-propanediol dehydrogenase activity, such as dhaT. In various instances one or more of the nucleic acids above are heterologous. In some instances, one or more nucleic acids are mutated for improved or increased activity. In some instances, one or more nucleic acids have been evolved. In some instances, one or more nucleic acids have been introduced to the microorganism by one or more vectors, such as a plasmid. In some instances, the microorganism biosynthesizes 1,4-BDO utilizing one or more of the gene products of the foregoing nucleic acids.

In some instances, the present invention contemplates a modified or recombinant microorganism comprising more than one of the foregoing nucleic acids. In some instances, the present invention contemplates a modified or recombinant microorganism comprising two of the foregoing nucleic acids. In some instances, the present invention contemplates a modified or recombinant microorganism comprising three of the foregoing nucleic acids. In some instances, the present invention contemplates a modified or recombinant microorganism comprising four of the foregoing nucleic acids. In some instances, the present invention contemplates a modified or recombinant microorganism comprising five of the foregoing nucleic acids. In some instances, the present invention contemplates a modified or recombinant microorganism comprising six of the foregoing nucleic acids.

In some instances, the present invention contemplates a modified or recombinant microorganism comprising more than one of the foregoing polypeptides. In some instances, the present invention contemplates a modified or recombinant microorganism comprising two of the foregoing polypeptides. In some instances, the present invention contemplates a modified or recombinant microorganism comprising three of the foregoing polypeptides. In some instances, the present invention contemplates a modified or recombinant microorganism comprising four of the foregoing polypeptides. In some instances, the present invention contemplates a modified or recombinant microorganism comprising five of the foregoing polypeptides. In some instances, the present invention contemplates a modified or recombinant microorganism comprising six of the foregoing polypeptides. In some instances, the microorganism biosynthesizes 1,4-BDO utilizing one or more of the foregoing polypeptides.

In some instances, the present invention contemplates a modified or recombinant microorganism that is adapted to biosynthesize 1,4-BDO by condensing two acetyl-CoA moieties into acetoacetyl-CoA.

In some instances, the present invention contemplates a modified or recombinant microorganism comprising aldehyde dehydrogenase.

In some instances, the present invention contemplates a genetically modified (recombinant) microorganism comprising one or more nucleic acid sequences that encodes one or more polypeptides with at least 85%, 90%, 95%, 99% or 100% amino acid sequence identity to any of the enzymes of any of 1,4-BDO biosynthetic pathway A, wherein the one or more polypeptides have enzymatic activity effective to perform the enzymatic reaction of the respective 1,4-BDO biosynthetic pathway enzyme, and the recombinant microorganism biosynthesizes 1,4-BDO. For example, in one instance, the present invention contemplates a modified or recombinant microorganism comprising a nucleic acid encoding a polypeptide having α-ketoglutarate decarboxylase activity, such as kgd. In another instance, the present invention contemplates a modified or recombinant microorganism comprising a nucleic acid encoding a polypeptide having 4-hydroxybutyrate dehydrogenase activity, such as 4hbD. In another instance, the present invention contemplates a modified or recombinant microorganism comprising a nucleic acid encoding a polypeptide having 1,3-propanediol dehydrogenase activity, such as dhaT. In various instances one or more of the nucleic acids above are heterologous. In some instances, one or more nucleic acids are mutated for improved or increased activity. In some instances, one or more nucleic acids have been evolved. In some instances, one or more nucleic acids have been introduced to the microorganism by one or more vectors, such as a plasmid. In some instances, the microorganism biosynthesizes 1,4-BDO utilizing one or more of the gene products of the foregoing nucleic acids. In some instances, the present invention contemplates a modified or recombinant microorganism comprising more than one of the foregoing nucleic acids. In some instances, the present invention contemplates a modified or recombinant microorganism comprising two of the foregoing nucleic acids. In some instances, the present invention contemplates a modified or recombinant microorganism comprising three of the foregoing nucleic acids.

In some instances, the present invention contemplates a modified or recombinant microorganism comprising more than one of the foregoing polypeptides. In some instances, the present invention contemplates a modified or recombinant microorganism comprising two of the foregoing polypeptides. In some instances, the present invention contemplates a modified or recombinant microorganism comprising three of the foregoing polypeptides. In some instances, the microorganism biosynthesizes 1,4-BDO utilizing one or more of the foregoing polypeptides.

In some instances, the present invention contemplates a modified or recombinant microorganism that is adapted to biosynthesize 1,4-BDO from citrate, wherein the citrate is derived from oxaloacetate and acetyl-CoA. In some instances, the recombinant microorganism comprises aconitase, isocitrate dehydrogenase, aldehyde dehydrogenase, and methylcitrate synthase. In some instances, the recombinant microorganism comprises aconitase, isocitrate dehydrogenase, aldehyde dehydrogenase, and citrate synthase.

In some instances, the present invention contemplates a genetically modified (recombinant) microorganism comprising one or more nucleic acid sequences that encodes one or more polypeptides with at least 85%, 90%, 95%, 99% or 100% amino acid sequence identity to any of the enzymes of any of 1,4-BDO biosynthetic pathway C, wherein the one or more polypeptides have enzymatic activity effective to perform the enzymatic reaction of the respective 1,4-BDO biosynthetic pathway enzyme, and the recombinant microorganism biosynthesizes 1,4-BDO. For example, in one instance, the present invention contemplates a modified or recombinant microorganism comprising a nucleic acid encoding a polypeptide having fumarase activity, such as fumA, fumB, or fumC. In another instance, the present invention contemplates a modified or recombinant microorganism comprising a nucleic acid encoding a polypeptide having fumarate reductase activity, such as frd. In another instance, the present invention contemplates a modified or recombinant microorganism comprising a nucleic acid encoding a polypeptide having succinate semialdehyde dehydrogenase activity, such as yneI. In another instance, the present invention contemplates a modified or recombinant microorganism comprising a nucleic acid encoding a polypeptide having one or both of succinyl-CoA synthetase activity and succinate semialdehyde dehydrogenase activity, such as sucC and/or sucD. In another instance, the present invention contemplates a modified or recombinant microorganism comprising a nucleic acid encoding a polypeptide having 4-hydroxybutyrate dehydrogenase activity, such as 4hbD. In another instance, the present invention contemplates a modified or recombinant microorganism comprising a nucleic acid encoding a polypeptide having aldehyde dehydrogenase activity, such as adh. In another instance, the present invention contemplates a modified or recombinant microorganism comprising a nucleic acid encoding a polypeptide having 1,3-propanediol dehydrogenase activity, such as dhaT. In various instances one or more of the nucleic acids above are heterologous. In some instances, one or more nucleic acids are mutated for improved or increased activity. In some instances, one or more nucleic acids have been evolved. In some instances, one or more nucleic acids have been introduced to the microorganism by one or more vectors, such as a plasmid. In some instances, the microorganism biosynthesizes 1,4-BDO utilizing one or more of the gene products of the foregoing nucleic acids.

In some instances, the present invention contemplates a modified or recombinant microorganism comprising more than one of the foregoing nucleic acids. In some instances, the present invention contemplates a modified or recombinant microorganism comprising two of the foregoing nucleic acids. In some instances, the present invention contemplates a modified or recombinant microorganism comprising three of the foregoing nucleic acids. In some instances, the present invention contemplates a modified or recombinant microorganism comprising four of the foregoing nucleic acids. In some instances, the present invention contemplates a modified or recombinant microorganism comprising five of the foregoing nucleic acids. In some instances, the present invention contemplates a modified or recombinant microorganism comprising six of the foregoing nucleic acids. In some instances, the present invention contemplates a modified or recombinant microorganism comprising seven of the foregoing nucleic acids.

In some instances, the present invention contemplates a modified or recombinant microorganism comprising more than one of the foregoing polypeptides. In some instances, the present invention contemplates a modified or recombinant microorganism comprising two of the foregoing polypeptides. In some instances, the present invention contemplates a modified or recombinant microorganism comprising three of the foregoing polypeptides. In some instances, the present invention contemplates a modified or recombinant microorganism comprising four of the foregoing polypeptides. In some instances, the present invention contemplates a modified or recombinant microorganism comprising five of the foregoing polypeptides. In some instances, the present invention contemplates a modified or recombinant microorganism comprising six of the foregoing polypeptides. In some instances, the present invention contemplates a modified or recombinant microorganism comprising seven of the foregoing polypeptides. In some instances, the microorganism biosynthesizes 1,4-BDO utilizing one or more of the foregoing polypeptides.

In some instances, the present invention contemplates a modified or recombinant microorganism that is adapted to biosynthesize 1,4-BDO from malate, wherein the malate is derived from oxaloacetate and/or from pyruvate. In some instances, the recombinant microorganism comprises fumarase, succinate semialdehyde dehydrogenase, and aldehyde dehydrogenase. In some instances, the recombinant microorganism comprises fumarase, succinyl-CoA synthetase, and aldehyde dehydrogenase.

In some instances, the present invention contemplates a recombinant microorganism comprising any nucleic acid disclosed herein, wherein the nucleic acid molecule selectively hybridizes with any one of the nucleic acid sequences of SEQ ID NOs 0001-0007, and 0012 or one that is at least 50, 60, 70, 80, 90, 95 or 99% homologous thereto.

A recombinant microorganism comprising all enzyme functions for one, for two, or for all three of the above 1,4-BDO biosynthetic pathways.

Any of the above recombinant microorganisms that additionally comprise genetic elements that provide increased tolerance to 1,4-BDO (whether naturally occurring or introduced by genetic modifications).

The above paragraphs are meant to indicate modifications in the nucleic acid sequences may be made and a respective polypeptide encoded there from remains functional so as to perform an enzymatic catalysis along one of the 1,4-BDO biosynthetic pathways A, B or C.

Also, and more generally, in accordance with examples and embodiments herein, there may be employed conventional molecular biology, cellular biology, microbiology, and recombinant DNA techniques within the skill of the art. Such techniques are explained fully in the literature. (See, e.g., Sambrook and Russell, Molecular Cloning: A Laboratory Manual, Third Edition 2001 (volumes 1-3), Cold Spring Harbor Laboratory Press, Cold Spring Harbor, N.Y.; Animal Cell Culture, R. I. Freshney, ed., 1986). These published resources are incorporated by reference herein for their respective teachings of standard laboratory methods found therein. Further, all patents, patent applications, patent publications, and other publications referenced herein (collectively, “published resource(s)”) are hereby incorporated by reference in this application. Such incorporation, at a minimum, is for the specific teaching and/or other purpose that may be noted when citing the reference herein. If a specific teaching and/or other purpose is not so noted, then the published resource is specifically incorporated for the teaching(s) indicated by one or more of the title, abstract, and/or summary of the reference. If no such specifically identified teaching and/or other purpose may be so relevant, then the published resource is incorporated in order to more fully describe the state of the art to which the present invention pertains, and/or to provide such teachings as are generally known to those skilled in the art, as may be applicable. However, it is specifically stated that a citation of a published resource herein shall not be construed as an admission that such is prior art to the present invention.

While various embodiments of the present invention have been shown and described herein, it will be obvious that such embodiments are provided by way of example only. Numerous variations, changes and substitutions may be made without departing from the invention herein in its various embodiments. Specifically, and for whatever reason, for any grouping of compounds, nucleic acid sequences, polypeptides including specific proteins including functional enzymes, metabolic pathway enzymes or intermediates, elements, or other compositions, or concentrations stated herein in a list, table, or other grouping, unless clearly stated otherwise, it is intended that each such grouping provides the basis for and serves to identify various subset embodiments, the subset embodiments in their broadest scope comprising every subset of such grouping by exclusion of one or more members of the respective stated grouping. Moreover, when any range is described herein, unless clearly stated otherwise, that range includes all values therein and all sub-ranges therein. Accordingly, it is intended that the invention be limited only by the spirit and scope of appended claims, and of later claims, and of either such claims as they may be amended during prosecution of this or a later application claiming priority hereto.

What is claimed is:

1-31. (canceled)

32. A recombinant microorganism comprising nucleic acid sequences encoding polypeptides that convert 4-hydroxybutyrate (“4-HB”) to 4-hydroxybutanal and 4-hydroxybutanal to 1,4-butanediol (“1,4-BDO”), thereby biosynthesizing 1,4-BDO from carbon sources provided to the recombinant microorganism.

33. The recombinant microorganism of claim 32 comprising nucleic acid sequences encoding polypeptides for at least one of the following pathways:
a. a pathway that converts citrate to cis-aconitate to D-isocitrate to alpha ketoglutarate to succinate semialdehyde to the 4-HB;
b. a pathway that converts acetyl-CoA to acetoacetyl-CoA to 3-hydroxybutryryl-CoA to crotonyl-CoA to vinylacetyl-CoA to 4-hydroxybutyryl-CoA to the 4-HB; or
c. a pathway that converts succinate to succinate semialdehyde (optionally including via succinyl-CoA) to the 4-HB;
thereby providing 4-HB.

34. A recombinant microorganism comprising a biosynthetic pathway with aconitase, isocitrate dehydrogenase, and alpha ketoglutarate decarboxylase enzymatic activities.

35. The recombinant microorganism of claim 34 additionally comprising 4-hydroxybutyrate dehydrogenase, aldehyde dehydrogenase, and 1,3 propanediol dehydrogenase enzymatic activities.

36. The recombinant microorganism of claim 34, additionally comprising a biosynthetic pathway with acetyl-coA acetyltransferase, beta-hydroxybutyryl-coA dehydrogenase, crotonase, vinylacetyl-coA-isomerase, 4-hydroxybutyryl-coA dehydratase, and 4-hydroxybutyrate coA transferase enzymatic activities.

37. The recombinant microorganism of claim 34, additionally comprising a biosynthetic pathway with succinate semialdehyde dehydrogenase, and optionally with succinyl-coA synthetase, enzymatic activities.

38. A recombinant microorganism comprising a biosynthetic pathway with acetyl-coA acetyltransferase, beta-hydroxybutyryl-coA dehydrogenase, crotonase, vinylacetyl-coA-isomerase, 4-hydroxybutyryl-coA dehydratase, and 4-hydroxybutyrate coA transferase enzymatic activities.

39. The recombinant microorganism of claim 38, additionally comprising 4-hydroxybutyrate dehydrogenase, aldehyde dehydrogenase, and 1,3 propanediol dehydrogenase enzymatic activities.

40. A recombinant microorganism of claim 38, additionally comprising a biosynthetic pathway with succinate semialdehyde dehydrogenase, and optionally with succinyl-coA synthetase, enzymatic activities.

41. A recombinant microorganism of claim 40, comprising modifications in nucleic acids encoding respective polypeptides of the biosynthetic pathways, and resulting in biosynthesis of 3-hydroxybutyryl-coA.

42. A recombinant microorganism comprising a biosynthetic pathway with succinate semialdehyde dehydrogenase, and optionally with succinyl-coA synthetase, enzymatic activities.

43. The recombinant microorganism of claim 42 additionally comprising 4-hydroxybutyrate dehydrogenase, aldehyde dehydrogenase, and 1,3 propanediol dehydrogenase enzymatic activities.

44. A method of biosynthesis of a polymer chemistry intermediate comprising providing in a bioreactor vessel a recombinant microorganism of claim 34, a carbon source, and a media, and conducting a bio-production event under suitable conditions and for a suitable time to obtain a measurable quantity of the intermediate.

45. The method of claim 44, wherein the intermediate is 1,4-BDO.

46. A method of making a polyester, a polyurethane, butyrolactone, γ-butyrolactone, or tetrahydrofuran comprising converting the 1,4-BDO of claim 45 to a polyester, a polyurethane, butyrolactone, γ-butyrolactone, or tetrahydrofuran.

47. A method of biosynthesis of a polymer chemistry intermediate comprising providing in a bioreactor vessel a recombinant microorganism of claim 38, a carbon source, and a media, and conducting a bio-production event under suitable conditions and for a suitable time to obtain a measurable quantity of the intermediate.

48. The method of claim 47, wherein the intermediate is 1,4-BDO.

49. A method of making a polyester, a polyurethane, butyrolactone, γ-butyrolactone, or tetrahydrofuran comprising converting the 1,4-BDO of claim 48 to a polyester, a polyurethane, butyrolactone, γ-butyrolactone, or tetrahydrofuran.

50. A method of biosynthesis of a polymer chemistry intermediate comprising providing in a bioreactor vessel a recombinant microorganism of claim 42, a carbon source, and a media, and conducting a bio-production event under suitable conditions and for a suitable time to obtain a measurable quantity of the intermediate.

51. The method of claim 50, wherein the intermediate is 1,4-BDO.

52. A method of making a polyester, a polyurethane, butyrolactone, γ-butyrolactone, or tetrahydrofuran comprising converting the 1,4-BDO of claim 51 to a polyester, a polyurethane, butyrolactone, γ-butyrolactone, or tetrahydrofuran.

53. An industrial-scale microbial bioreactor system comprising:
(a) a bioreactor vessel;
(b) a carbon source;
(c) a recombinant microorganism of claim 32; and
(d) a media;
wherein the carbon source is optionally selected from the group consisting of sucrose, glucose, xylose, cellulose or hemicelluloses; and
wherein the media is optionally a minimal media; and further wherein said minimal media optionally comprises M9 minimal media, potassium sulfate minimal media, yeast synthetic minimal media and variations thereof.

54. The industrial-scale microbial bioreactor system of claim 53, wherein the recombinant microorganism of claim 1 (step (c)) additionally comprises nucleic acid sequences encoding polypeptides for at least one of the following pathways:
a. a pathway that converts citrate to cis-aconitate to D-isocitrate to alpha ketoglutarate to succinate semialdehyde to the 4-HB;
b. a pathway that converts acetyl-CoA to acetoacetyl-CoA to 3-hydroxybutryryl-CoA to crotonyl-CoA to vinylacetyl-CoA to 4-hydroxybutyryl-CoA to the 4-HB; or
c. a pathway that converts succinate to succinate semialdehyde (optionally including via succinyl-CoA) to the 4-HB;
thereby providing 4-HB in the recombinant microorganism.
